(19) United States
(12) Patent Application Publication
(10) Pub. No.: US 2022/0167950 A1
(43) Pub. Date: Jun. 2, 2022

(54) SYSTEMS AND METHODS FOR PROCESSING STOOL SAMPLES

(71) Applicant: BiomeSense, Inc., Chicago, IL (US)

(72) Inventors: Dylan Nichols, Chicago, IL (US); Kevin Honaker, Chicago, IL (US); Navid Ghorashian, Chicago, IL (US); Thao Dang, Oak Lawn, IL (US)

(21) Appl. No.: 17/552,702

(22) Filed: Dec. 16, 2021

Related U.S. Application Data
(63) Continuation of application No. PCT/US2020/038563, filed on Jun. 18, 2020.
(60) Provisional application No. 62/864,419, filed on Jun. 20, 2019.

(51) Int. Cl. A61B 10/00; A61B 5/00

(52) U.S. Cl. CPC A61B 10/0038; A61B 5/0002; B01L 2300/023; B01L 3/502753; B01L 2300/0883; B01L 2400/043; B01L 2400/0655; B01L 2300/0829

(57) ABSTRACT

Methods and systems directed to collecting, processing, and analyzing stool samples are disclosed herein. A stool sample may be collected from a subject using a stool collector and collected in a collection unit. The stool sample may be analyzed using a sensor. The sensor may be used to determine one or more parameters of the stool sample, e.g., for nucleic acid or protein analysis. One or more biomolecules (e.g., proteins, nucleic acids) may be extracted or isolated from the stool sample using the methods and systems disclosed herein.

### CROSS-REFERENCE

This application is a continuation application of International Application No. PCT/US2020/038563, filed Jun. 18, 2020, which claims the benefit of U.S. Provisional Patent Application No. 62/864,419, filed Jun. 20, 2019, each of which is incorporated by reference herein in its entirety.

### BACKGROUND

A sample may be collected for various purposes, such as identification of a property of the sample. The sample may be a biological sample or biology-derived sample. Biological samples may be processed, such as for disease detection and diagnosis, identification of contaminants or screening. Various approaches for processing samples may be performed, such as polymerase chain reaction (PCR) and sequencing, or using enzymatic assays.

Biological samples may be collected using a variety of approaches, such as physical capture. Devices or systems may be employed to capture or collect a biological sample that enables further analysis of the biological sample.

Biological samples may be subjected to various processes, such as chemical or physical processes. Samples may be subjected to heating or cooling, chemical reactions, mechanical reactions, such as to yield a sample or species that can processed qualitatively or quantitatively.

### SUMMARY

In an aspect, disclosed herein is a method for processing a stool sample, comprising: (a) receiving the stool sample at a first location, wherein the stool sample is collected at a second location different than the first location, and wherein the second location is within 1 mile of the first location; and (b) at the first location, automatically processing the stool sample to extract or isolate at least one biomolecule from the stool sample.

In some embodiments, the method further comprises transmitting the at least one biomolecule of (b) or derivative thereof to a third location different than the first location and the second location, wherein at the third location, the at least one biomolecule or derivative thereof is analyzed to identify at least a portion of the at least one biomolecule. In some embodiments, the method further comprises analyzing the at least one biomolecule at the first location. In some embodiments, the at least one biomolecule comprises a deoxyribonucleic acid (DNA) molecule, and wherein the analyzing comprises sequencing the DNA molecule. In some embodiments, the at least one biomolecule is selected from the group consisting of a nucleic acid, a polypeptide, a protein, a lipid, a carbohydrate, a metabolite, and a combination thereof. In some embodiments, (b) comprises using one or more beads to extract or isolate the at least one biomolecule. In some embodiments, the one or more beads comprises one or more magnetic beads. In some embodiments, the stool sample is received and processed in a collection unit. In some embodiments, the collection unit comprises a sensing unit comprising a sensor and an electronics unit. In some embodiments, the method further comprises using the sensor to analyze the at least one biomolecule. In some embodiments, the electronics unit of the sensing unit comprises a communication interface for transmitting data of the stool sample or derivative thereof to an electronic device in communication with the communication interface. In some embodiments, the method further comprises automatically cleaning the collection unit. In some embodiments, the collection unit comprises a storage unit. In some embodiments, the storage unit comprises a multi-well unit, and wherein the at least one biomolecule is extracted or isolated in a well of the multi-well unit. In some embodiments, the storage unit comprises a plurality of cartridges. In some embodiments, the method further comprises storing the at least one biomolecule in the storage unit. In some embodiments, the storage unit is stored at ambient temperature. In some embodiments, the collection unit comprises an array, and wherein the at least one biomolecule is extracted or isolated using the array. In some embodiments, the method further comprises analyzing the at least one biomolecule on the array. In some embodiments, (b) comprises homogenizing the stool sample. In some embodiments, (b) comprises lysis of one or more cells in the stool sample. In some embodiments, the lysis is performed using one or more members selected from the group consisting of ultrasonic lysis, mechanical lysis, biological lysis, and chemical lysis. In some embodiments, the lysis comprises mechanical lysis. In some embodiments, the mechanical lysis is performed using beads. In some embodiments, (b) comprises filtering the stool sample or derivative thereof. In some embodiments, the method further comprises (c) automatically processing another stool sample to extract or isolate at least one biomolecule from the another stool sample. In some embodiments (b) and (c) are performed without involvement of a user.

In another aspect, provided herein is a method for processing a stool sample of a subject, comprising: (a) providing (i) a stool collector comprising a first housing coupled to a toilet and (ii) a collection unit comprising a second housing, wherein the second housing is coupled to the first housing; (b) using the stool collector to collect the stool sample of the subject; and (c) in the collection unit, extracting or isolating a biomolecule from the stool sample or derivative thereof.

In some embodiments, the collection unit is coupled to the stool collector through a fluid flow path. In some embodiments, the collection unit further comprises a sensor comprising an electronics unit. In some embodiments, the method further comprises using the sensor to analyze the biomolecule. In some embodiments, the electronics unit of the sensor comprises a communication interface for transmitting data of the stool sample or derivative thereof to an electronic device in communication with the communication interface. In some embodiments, the communication interface is a wireless communication interface. In some embodiments, the method further comprises, prior to (c), processing the stool sample to yield a processed stool sample. In some embodiments, the biomolecule is extracted or isolated from the processed stool sample. In some embodiments, the processing comprises homogenizing the stool sample. In some embodiments, the processing comprises lysis of one or more cells in the stool sample. In some embodiments, the processing comprises filtering the stool sample or derivative thereof. In some embodiments, the lysis is performed using one or more members selected from the group consisting of ultrasonic lysis, mechanical lysis, biological lysis, and chemical lysis. In some embodiments, the lysis comprises using mechanical lysis. In some embodiments, the mechanical lysis is performed using beads. In some embodiments, the method further comprises, following (c), automatically replacing the beads in the collection unit. In some embodiments, the method further comprises, following (c), automatically cleaning the beads in the collection unit. In some embodiments, the stool collector and the collection unit are in fluid communication with a lysis chamber, and wherein the processing occurs in the lysis chamber. In some embodiments, the method further comprises automatically cleaning the stool collector or the collection unit. In some embodiments, the stool collector or the collection unit is cleaned automatically without any involvement from a user. In some embodiments, the biomolecule is a nucleic acid, polypeptide or protein, lipid, carbohydrate, or metabolite. In some embodiments, wherein (c) comprises using one or more beads to perform the extracting or isolating. In some embodiments, the one or more beads comprises one or more magnetic beads. In some embodiments, the method further comprises imaging the stool sample or derivative thereof. In some embodiments, the collection unit comprises a storage unit. In some embodiments, the storage unit comprises a multi-well unit, and wherein the biomolecule is extracted or isolated in a well of the multi-well unit. In some embodiments, the method further comprises storing the biomolecule in the storage unit. In some embodiments, the storage unit is performed at ambient temperature. In some embodiments, the collection unit comprises an array, and wherein the biomolecule is extracted or isolated using the array. In some embodiments, the collection unit comprises an array, and wherein the biomolecule is analyzed using the array.

In yet another aspect, disclosed herein is a system for processing a stool sample of a subject, comprising: a stool collector configured to couple to a toilet; and a collection unit coupled or configured to couple to the stool collector, wherein the stool collector is configured to collect the stool sample of the subject, and wherein the collection unit is configured to extract or isolate a biomolecule from the stool sample or derivative thereof.

In some embodiments, the collection unit is coupled or configured to couple to the stool collector through a fluid flow path. In some embodiments, the collection unit further comprises a sensor comprising an electronics unit. In some embodiments, the sensor is configured to analyze the biomolecule. In some embodiments, the electronics unit of the sensor comprises a communication interface for transmitting data of the stool sample to an electronic device in communication with the communication interface. In some embodiments, the communication interface is a wireless communication interface.

In another aspect of the present disclosure, provided herein is a method for analyzing a stool sample of a subject, comprising: (a) providing (i) a stool collector coupled to a toilet and (ii) a sensing unit comprising a sensor comprising an electronics unit, wherein the sensing unit is coupled to the stool collector; (b) using the stool collector to collect the stool sample of the subject; (c) directing the stool sample or derivative thereof to the sensing unit; (d) using the electronics unit of the sensor to process the stool sample or derivative thereof, thereby yielding a processed stool sample; and (e) using the sensor to analyze (i) the processed stool sample or derivative thereof, or (ii) a chemical or biological material within the processed stool sample.

In some embodiments, the electronics unit comprises electrical inlets for applying an electric field to the processed stool sample or derivative thereof. In some embodiments, the chemical or biological material is a cell, virus, nucleic acid, polypeptide or protein, lipid, carbohydrate, small molecule, metal or metabolite. In some embodiments, the sensor comprises a microfluidic device. In some embodiments, the electronics unit of the sensor comprises a communication interface for transmitting data of the parameter to an electronic device in communication with the communication interface. In some embodiments, the communication interface is a wireless communication interface. In some embodiments, the wireless communication interface is a Wi-Fi interface. In some embodiments, the wireless communication interface is a near field communication interface. In some embodiments, the wireless communication interface is a Bluetooth interface. In some embodiments, the method further comprises imaging the processed stool sample or derivative thereof.

In yet another aspect, disclosed herein is a system for analyzing a stool sample of a subject, comprising: a stool collector configured to couple to a toilet; and a sensing unit comprising a sensor comprising an electronics unit, wherein the sensing unit is coupled to or configured to couple to the stool collector; wherein the stool collector is configured to collect the stool sample of the subject, wherein the sensing unit is configured to receive the stool sample or derivative thereof collected from the subject, wherein the electronics unit is configured to process the stool sample or derivative thereof, and wherein the sensor is configured to analyze (i) the stool sample or derivative thereof, or (ii) a chemical or biological material within the stool sample.

In some embodiments, the stool collector is coupled to or configured to couple to the sensing unit through a fluid flow path. In some embodiments, the electronics unit of the sensor comprises a communication interface for transmitting data of the stool sample to an electronic device in communication with the communication interface. In some embodiments, the communication interface is a wireless communication interface. In some embodiments, the electronics unit comprises electrical inlets for applying an electric field to the processed stool sample or derivative thereof.

In another aspect, disclosed herein is a method for analyzing a stool sample, comprising: (a) providing (i) a stool collector configured to couple to a toilet and (ii) a sensor comprising an electronics unit, wherein the sensor is configured to couple to the stool collector; (b) using the stool collector to collect a stool sample of a subject from a location within the toilet; (c) directing the stool sample to the sensor to yield a processed stool sample; (d) using the electronics unit of the sensor to analyze a parameter of the processed stool sample.

In some embodiments, the parameter comprises the presence of one or more molecules selected from the group consisting of microbial nucleic acids, human nucleic acids, viral nucleic acids, microbial cells, human cells, blood, metabolites, proteins, small molecules, and metals. In some embodiments, the sensor is part of a microfluidic device. In some embodiments, the sensor is coupled to a microfluidic device. In some embodiments, the electronics unit of the sensor comprises a communication interface for transmitting data of the parameter to an electronic device in communication with the communication interface. In some embodiments, the communication interface is a wireless communication interface. In some embodiments, the wireless communication interface is a Wi-Fi interface. In some embodiments, the wireless communication interface is a near field communication interface. In some embodiments, the wireless communication interface is a Bluetooth interface. In some embodiments, the method further comprises imaging the processed stool sample.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 schematically illustrates an example workflow for analyzing a stool sample. FIG. 1A shows an example workflow for analyzing a stool sample using an example sensor comprising a microfluidic device. FIG. 1B shows an example workflow for analyzing a stool sample using another example sensor and a collection unit.

FIG. 2 shows a schematic of a sensor to process a stool sample for analyzing a parameter of the stool sample or derivative thereof. FIG. 2A shows a schematic of a microfluidic device to analyze the stool sample. FIG. 2B shows a schematic of a microfluidic device comprising a plurality of features. FIG. 2C shows an alternate schematic of a microfluidic device comprising a plurality of features.

FIG. 3 schematically shows an example microfluidic device used for further processing of the stool sample.

FIG. 4 provides images of a microfluidic device used for analyzing a stool sample. FIG. 4A shows a microfluidic device coupled to a plurality of reservoirs. FIG. 4B shows a microfluidic device comprising a channel for processing and/or analyzing a stool sample. FIG. 4C shows a microfluidic device coupled to electrical components for processing and/or analyzing a stool sample.

FIG. 5 schematically shows an example microfluidic device used for analyzing a parameter of the stool sample.

FIG. 6 schematically shows an example system for analyzing a parameter of the processed stool sample.

FIG. 7 shows example data of the measured abundance of DNA in simulated stool samples.

FIG. 8 shows another example data of the measured abundance of DNA in simulated stool samples.

FIG. 9 schematically shows an example of a system comprising a stool collector and collection unit.

FIG. 10 shows an example of a stool collector.

FIG. 11 shows another example of a stool collector.

FIG. 12 shows yet another example of a stool collector.

FIG. 13 shows yet another example of a stool collector.

FIG. 14 shows an example of a collection unit. FIG. 14A shows an integrated collection unit. FIG. 14B shows an example housing of the collection unit. FIG. 14C shows an example housing and cover of the collection unit.

FIG. 15 shows a diagram of an example system architecture. FIG. 15A shows an example system architecture comprising a stool collector and a collection unit. FIG. 15B shows an enlarged view of the example architecture of the stool collector. FIG. 15C shows an enlarged view of the example architecture of the collection unit.

FIG. 16 schematically shows two examples of a lysis system, which may be included in the stool collector or the collection unit. FIG. 16A shows a lysis system that comprises a lysis chamber and a rotatable impeller. FIG. 16B illustrates schematically another example lysis system that also comprises a lysis chamber and a rotatable impeller.

FIG. 17 schematically illustrates an example mechanism for dispensing of beads into a lysis unit of the collection unit. FIG. 17A shows a perspective view of the bead dispensing unit. FIG. 17B shows a cross-sectional view of the bead dispensing unit in an open configuration. FIG. 17C shows a cross-sectional view of the bead dispensing unit in a closed configuration.

FIG. 18 shows an example storage unit in an example collection unit.

FIG. 19 shows example data of the measured abundance of an extracted biomolecule in stool samples.

FIG. 20 shows example data of the measured abundance of extracted biomolecules using sequencing.

FIG. 21 shows additional example data of the measured abundance of extracted biomolecules using sequencing.

FIG. 22 shows a computer system that is programmed or otherwise configured to implement methods provided herein.

FIG. 23 schematically shows an example controller architecture.

FIG. 24 schematically shows a microcontroller unit.

FIG. 25 schematically shows a controller comprising a stepper driver.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “real time,” as used herein, can refer to a response time of less than about 1 second, a tenth of a second, a hundredth of a second, a millisecond, or less. The response time may be greater than 1 second. In some instances, real time can refer to simultaneous or substantially simultaneous processing, detection or identification.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer), or a pre-disposition to the disease, an infection, and/or an individual that is in need of therapy or suspected of needing therapy. A subject can be a patient. A subject can be a microorganism or microbe (e.g., bacteria, fungi, archaea, viruses).

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA, complementary DNA, etc.). Sequencing can be performed using an array (e.g., a hybridization array), single-molecule sequencing, sequencing by synthesis, or sequencing by ligation. Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR) (e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject. In some examples, such systems provide sequencing reads (also “reads” herein). A read may include a string of nucleic acid bases corresponding to a sequence of a nucleic acid molecule that has been sequenced. In some situations, systems and methods provided herein may be used with proteomic information.

The term “sample,” as used herein, generally refers to a biological sample of a subject. The biological sample may comprise any number of macromolecules, for example, cellular macromolecules. The sample may be a stool sample. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from (e.g., processed from) another sample. The sample may be a stool sample or derived from a stool sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears.

### Stool Sample Collection and Processing

In an aspect, the present disclosure provides methods and systems for the collection, processing, and/or analysis of one or more samples. The sample may be collected from a subject, e.g., a human, and may comprise a stool sample, or derivative thereof. A method for analyzing a stool sample may comprise (a) providing: (i) a stool collector coupled to a toilet to collect a stool sample of a subject from a location within a toilet bowl and (ii) a sensing unit comprising a sensor comprising an electronics unit, which sensing unit is configured to couple to the stool collector, (b) using the stool collector to collect a stool sample from a subject, (c) directing the stool sample to the sensing unit, (d) using the electronics unit of the sensor to process the stool sample or derivative thereof, thereby yielding a processed stool sample and (e) using the sensor to analyze the processed stool sample or derivative thereof, of a chemical or biological material within the processed stool sample. In some instances, the method comprises: (a) providing: (i) a stool collector configured to couple to a toilet and (ii) a collection unit coupled to the stool collector; and (b) using the stool collector to collect a stool sample of a subject.

The stool collector may be coupled to a toilet or the stool collector may be a part of a toilet. In some cases, the stool collector may be coupled to a fluid flow path separate from the stool collector. The stool sample may be directed along the fluid flow path from the stool collector to yield a processed stool sample. The stool sample or processed stool sample may be directed to a sensor configured to couple to the toilet to analyze a parameter of the processed stool sample. Alternatively or in addition to, the stool sample or processed stool sample may be directed to a collection unit.

In some instances, the stool collector is configured to couple to a toilet. The stool collector may be attached to a toilet seat, which may be coupled to the toilet bowl. In some cases, the stool collector is attached to the toilet bowl. The stool collector may be removably coupled to the toilet. The stool collector may be mechanically coupled to the toilet using one or more fastening mechanisms, or the stool collector may be adhered to the toilet (e.g., using adhesive tape or glue).

The stool collector may comprise a catchment unit, which, in certain instances, is retractable. The catchment unit may comprise any useful geometry or set of features to facilitate capture or collection of the stool sample from the subject. The catchment unit may comprise a catchment appendage (e.g., “fingers”, scoop, brush, funnel, cup, bowl, net, sieve, plate, coils, tubes, planar substrate, etc.). For instance, the catchment unit may comprise one or more catchment arms or rods. The catchment unit may additionally comprise sub-features, such as topographical features (e.g., ridges, bumps, teeth, pegs, or topographical features of any geometry, texture, or orientation) to aid in collection of the sample. Alternatively or in addition to, the catchment unit may comprise one or more membranes. For instance, the catchment unit may comprise a pair of retractable arms with a membrane disposed between the two arms. The catchment unit may be used to collect the stool sample of a subject from a location of a toilet or toilet bowl. The catchment unit may be configured to extend towards the toilet bowl during sample collection. In some instances, the catchment arm may be configured to retract towards a chamber for sample processing. The extension and/or retraction of the catchment unit or arm may be initiated automatically. In some cases, the extension may be initiated using one or more signals from a sensor. Alternatively or in addition to, the retraction of the catchment unit may be initiated using a signal from a sensor of the stool collector. The extension of the catchment unit may be performed using a variety of approaches, e.g., mechanical, electrical, or other approaches. For example, the stool collector may comprise a plurality of gears that can interface with a motor. The stool collector may be coupled to a motor, valve, or other useful part.

One or more motors may be coupled to one or more parts of the stool collector. The stool collector may comprise any suitable type of motor, e.g., an electric motor, a manual motor, an air motor, etc. In some instances, the motors may be coupled to one or more gears and/or one or more pumps. One or more motors may be used for a variety of purposes, including but not limited to: opening and/or closing or the homogenization chamber, extension and/or retraction of the catchment unit, homogenization of the stool sample, pumping a fluid or reagent into the homogenization chamber, directing the stool sample or processed stool sample (e.g., homogenized stool sample) away from the toilet and/or to a collection unit.

The stool sample may be processed to yield a derivative of the stool sample (e.g., processed stool sample). In some cases, the processing comprises homogenization of the stool sample. The stool collector may comprise or be coupled to a homogenization chamber, which may be a part of or coupled (e.g., via a fluid flow path) to the collection unit. The homogenization chamber may be a vessel. The homogenization chamber may be coupled to one or more fluid inlets or fluid flow paths. The stool sample may be directed to and/or deposited into the homogenization chamber (e.g., via retractable catchment fingers). During or following depositing of the stool sample into the homogenization chamber, one or more reagents (e.g., filtered water from the tank, buffers, sample processing solutions, etc.) may be introduced into the homogenization chamber (e.g., via the fluid inlet). The homogenization chamber may be used to homogenize the stool sample. For instance, the homogenization chamber may comprise blades coupled to a motor. The stool sample may be deposited into the homogenization chamber, and a homogenization buffer may be introduced (e.g., filtered water). The stool sample may be homogenized by actuation of the motor coupled to one or more blades to stir, blend, rotate, spin, move, mix, and/or homogenize the sample. In another example, the homogenization chamber may comprise one or more pumps (e.g., a centrifugal pump, displacement pump, rotary pump, piston pump, rotary lobe pump, rotary gear pump, diaphragm pump, screw pump, gear pump, vane pump, peristaltic pump, or a combination thereof) to generate a pressure (e.g., water pressure) or shear force to homogenize the sample (e.g., via mixing, vortexing, etc.). The homogenization chamber may comprise a homogenizer, e.g., a rotor stator homogenizer, mechanical mortar, blender, masher, mills (e.g., bead mills), rollers, mixers, and/or beads (e.g., solid beads, such as magnetic, metal, ceramic, or plastic beads). One or more forces (e.g., centrifugal, gravitational, positive or negative pressure, ultrasonic, magnetic etc.) may be applied to the sample, reagents, and/or other homogenizer materials (e.g., applying a magnetic force to magnetic beads) to homogenize the sample. Alternatively or in addition to, the homogenization chamber may comprise a heating or cooling element, and the sample may be heated or cooled to homogenize the sample. In some instances, a combination of approaches are used to homogenize the sample; for instance, water pressure may be used to generate shear force, which may be combined with beads for mechanically homogenizing the sample.

In some cases, the stool sample or the homogenized stool sample may be subjected to filtering. For example, the stool sample or derivative thereof may be subjected to conditions sufficient to filter the sample using gravitational filtration, centrifugal filtration, filter stacking, sedimentation, passive filtering, or filtration using a mesh, membrane or other filtration mechanism. A filter may comprise a membrane, beads, diaphragms, colloids, weir filters, pillar filters, cross-flow filters, solvent filters, sieves, or any other filter. In some cases, the system (e.g., stool collector, collection unit, or other part of the system) may comprise geometric features that may be used to filter the stool sample or derivative thereof. The geometric features may be any useful geometry or size for filtration. The geometric features may comprise one or more geometries, e.g., pillars, pyramids, pegs, cylinders, spheres, boxes. The geometric features may comprise one or more sizes. In some cases, the geometric features comprise micropillars that may decrease in size across one or more dimensions (e.g., length, width, height, or a combination thereof) to generate a gradient of size filters. In some cases, the geometric features comprise micropillars that decrease in size across one or more dimensions to generate discrete zones of size filters. The filters may be used to filter out debris or particles larger than a specified threshold.

The stool sample, or the homogenized stool sample (or filtered stool sample), may be directed to a sensor, a collection unit, or both, for further processing, e.g., to yield a processed stool sample. The stool sample may be directed along a fluid flow path to yield the processed stool sample, or the fluid flow path may direct the stool sample or processed stool sample to a sensor for further processing of the stool sample (or processed stool sample). The sensor may comprise an electronics unit that is used to further process the stool sample and/or analyze a parameter of the stool sample, as described elsewhere herein. In one non-limiting example, the electronics unit of the sensor comprises electrical inlets or couplings that allow for application of an electric field to the stool sample (or processed stool sample), which may help in further homogenization of the sample or lysing of cells within the sample, as is described below.

The stool collector may be coupled to and/or comprise one or more fluid inlets and outlets. A fluid inlet may be used to introduce one or more reagents to a portion of the stool collector (e.g., the homogenization chamber). For example, the fluid inlet may be used to introduce water, a buffer, or other reagent into the portion of the stool collector. The one or more reagents may be introduced to the stool collector via a coupled fluid flow path. The fluid inlet of the stool collector or portion thereof (e.g., homogenization chamber) may be coupled to a fluid flow path, e.g., via a hose, pipe, funnel, etc. The fluid inlet may be coupled to or in fluid communication with a source of reagents (e.g., water, buffers, sterilization solutions, etc.). The fluid inlet may comprise one or more valves (e.g., y-valve, y-tube, T-valve, etc.) that may control the flow volume or rate of the reagents into the portion of the stool collector. In some instances, the stool collector is coupled to and/or further comprises a fluid outlet, which may be used to direct the stool sample or processed stool sample away from the stool collector to be collected, processed, and/or analyzed. The sample outlet may be used to direct the stool sample to a collection unit or to a sensor for sample analysis.

In some instances, the fluid flow path may comprise a plurality of valves that may be used to control the flow volume or rate of a fluid into or out of the stool collector, or portion thereof. In some cases, it may be useful to have a valve that can interface with more than one inlet or outlet or portions of the fluid flow path. In such cases, a custom-built valve may be used. For example, a 3-way luer valve may be used. The 3-way luer valve may interface with a custom-built base. The custom-built base may accommodate one or more gears which may interface with the valves (e.g., 3-way luer valve) and another part, e.g., a motor. Use of such a custom-built valve may allow for programmable and optionally automated 3-way valving, obviating the need for solenoids.

The fluid flow path may be coupled to the stool collector. The coupling of the fluid flow path to the stool collector may be achieved using a pump. The pump may move the reagents to the fluid flow path. In some cases, the stool collector may comprise a pump connected to the fluid flow path and the stool collector. The pump may be a rotary pump, gear pump, piston pump, diaphragm pump, screw pump, vane pump, peristaltic pump, centrifugal pump, etc., or variations or a combination thereof. In some cases, the reagents may be directed to the fluid flow path using an applied or generated force, e.g., hydraulic, centrifugal, gravitational, frictional, tensional, spring, pneumatic, etc. One or more parts may be coupled to a power supply. Alternatively or in addition to, one or more parts may be configured to couple to an electrical outlet.

Following collection of the stool sample from the subject, one or more reagents may be used for sterilization of a part of the system (e.g., the stool collector, toilet bowl, etc.). In such cases, the sterilization reagents may comprise any suitable sterilization reagent, e.g., bleach, chlorine, antimicrobial agents, alcohol, an acid, a base, minerals, soap, etc. or a combination thereof. Sterilization of the stool collector may be performed automatically or manually.

In some instances, cleaning or sterilization comprises introduction of a sterilization reagent (e.g., bleach, chlorine, antimicrobials, etc.) to one or more parts of the stool collector. In some cases, the cleaning or sterilization reagent may be introduced to the stool collector, or portion thereof, through one of the fluid inlets. In some instances, a pump may be used to direct the sterilization reagent to the stool collector, or portion thereof. In some cases, the sterilization reagent may be used to clean the stool collector, or portion thereof, and the toilet bowl, or portion thereof. In some cases, the sterilization reagent may be introduced to the stool collector, or portion thereof, via spraying, pressure, vapor deposition, pumping, flooding, immersion, or other dispersion technique. The sterilization of the stool collector, or portion thereof, may occur automatically. For example, following stool collection, the stool sample may be deposited into a chamber (e.g., for homogenization and/or stool processing). Following the deposit of the stool sample, the sterilization may be initiated (e.g., via feedback control system). Alternatively or in addition to, a sensor may be used to detect when sterilization is needed. For example, an optical sensor may be used to detect stool specimens on the stool collector or portion thereof, thereby initiating sterilization. In another example, a weight sensor (e.g., on the catchment unit) may be used to provide an input for a sterilization control mechanism. Alternatively or in addition to, a timer may be integrated into the stool collector, such that sterilization is programmed to occur at a given or programmed frequency (e.g., once per minute, once per hour, once per day). In some cases, the subject may be able to initiate the sterilization. In some cases, the sterilization may be manually performed.

The stool collector may comprise a communication interface that allows for transmitting and/or receiving data corresponding from a portion of the stool collector (e.g., from a sensor). Alternatively or in addition to, the communication interface may be used for transmitting data on a parameter of the stool collector, e.g., indicators of sample collection, errors in the system, commands entered or executed by the stool collector, etc. In some cases, the data may be transmitted to an electronic device in communication with the communication interface. The communication interface may be a wireless communication interface, e.g., a Wi-Fi interface, a near-field communication interface, or a Bluetooth interface. The electronic device may be a device that may communicate with the communication interface. The electronic device may be a mobile device (e.g., a smart phone, tablet, laptop, etc.). Alternatively or in addition to, the communication interface may be a wired communication interface. The stool collector may comprise a port for communication and/or a power supply (e.g., universal serial bus (USB), USB-type C, Thunderbolt, etc.).

One or more processes described herein may be performed automatically. For instance, the stool collector may be configured to perform automated sample collection. Methods and systems described herein may allow for stool collection that does not require a subject to interact with the stool sample. In one non-limiting example, the stool collector may comprise a sensor or be coupled to a sensor that detects the presence of a subject (or user) or a sample in proximity to the sensor or stool collector. For instance, the stool collector may comprise a sensor that can sense a vibration, motion, temperature, pressure, or an optical parameter (e.g., colors, shadows, light attenuation, etc.), indicating that a subject is present near the stool collector. In such cases, and in instances where the stool collector is retractable, the stool collector may automatically extend toward the toilet bowl for catchment or collection of the stool sample when the subject is present or in proximity to the stool collector. Following collection of the stool sample, the stool collector may retract, thereby depositing the collected sample to a collection unit (e.g., chamber). In some instances, the stool collector may comprise a sensor, e.g., weight or optical sensor, that may detect when the stool sample has been deposited on the stool collector; accordingly, the stool collector may automatically move or otherwise direct the stool sample to a collection and/or processing unit (e.g., for sample homogenization).

Alternatively, or in addition to, one or more processes described herein may be performed manually. For instance, the stool collector may be manually powered on or activated by the subject prior to or during use (e.g., using a button, switch, or other actuator), or the stool collector may comprise a communication interface that communicates with a device (e.g., mobile phone, tablet, or computer) of the subject. Prior to use, the subject may use the device or otherwise provide a user input (e.g., by providing a signal through the device to the stool collector for activation). Following collection of the stool sample, the user may provide an input or signal that collection is complete (e.g., via manual activation or through an electronic interface), and the stool collector may retract, thereby depositing the collected sample to a collection unit (e.g., chamber). The collected stool sample may then be subjected to further processing or analysis.

Additional examples of systems and methods for collecting and/or processing a stool sample may include, for example, the systems and methods disclosed in U.S. patent application Ser. No. 16/902,795, filed Jun. 16, 2020, which is incorporated by reference herein in its entirety.

### Stool Sample Processing and Analysis

Disclosed herein, in certain embodiments, is a method for analyzing a stool sample of a subject comprising: (a) providing: (i) a stool collector coupled to or configured to couple to a toilet to collect a stool sample of a subject from a location within a toilet bowl and (ii) a sensing unit comprising a sensor comprising an electronics unit that is coupled to or configured to couple to the stool collector, (b) using the stool collector to collect a stool sample from the subject, (c) directing the stool sample or a derivative thereof to the sensing unit; (d) using the electronics unit of the sensor to process the stool sample or derivative thereof to generate a processed stool sample; and (e) using the sensor to analyze the processed stool sample, such as a parameter of the processed stool sample, or a chemical or biological material within the processed stool sample.

The sensor comprising the electronics unit may be configured to couple to the stool collector. In some cases, the sensor may comprise a housing that is configured to couple to the stool collector. The housing may be attached to the stool collector, which may be attached to a toilet. The housing may be removably coupled to the stool collector and/or the toilet. The sensor may be mechanically coupled to the stool collector using one or more fastening mechanisms. For example, the stool collector may comprise threads (e.g., screw threads, internal threads) and the sensor may comprise complementary threads that may engage with the threads of the stool collector. Alternatively or in addition to, the stool collector may comprise snap-fit joints (e.g., cantilever snap fits, annular snap fits, etc.) that allow for interlocking of the stool collector to the sensor. Other interlocking or form-fitting pairs may be used to secure the stool collector to the sensor, e.g., hook and loop, latches, snap-ons, buttons, nuts and bolts, screws, magnets, etc. Alternatively or in addition to, the stool collector may comprise components that allow for fitting into the sensor, e.g., via an interference fit, force fit, shrink fit, location fit, etc. Other fastening mechanisms are possible, such as form-fitting pairs, hooks and loops, latches, threads, screws, staples, clips, clamps, prongs, rings, brads, rubber bands, rivets, grommets, pins, ties, snaps, Velcro, vacuum, seals, or a combination thereof. The stool collector may be adhered to the toilet (e.g., using adhesive tape or glue). In some instances, the sensor may be a part of the stool collector (i.e., integrated) and may not be removable from the stool collector.

In some instances, the sensor may be connected to the stool collector and/or toilet via an external part. The sensor may be connected to the stool collector via a fluid flow path. The part or fluid flow path may comprise any useful connector such as tubing, vessels (e.g., tubes, containers, vials, flasks), valves, clamps, clips, funnels, etc. In some cases, the stool sample or derivative thereof (e.g., homogenized sample) may be directed to the sensor via tubing and one or more pumps. For example, following the collection of the stool sample or derivative thereof (e.g., homogenized stool sample), the stool sample or derivative thereof may be directed to a container or collection unit, which may be coupled to the sensor via tubing and a pump. In such cases, the stool sample or derivative thereof may be pumped through the tubing to the sensor. The stool sample may be directed to the sensor via one or more forces, e.g., positive pressure, negative pressure (e.g., aspiration), centrifugal, capillary, gravitational, frictional, electric, magnetic forces. In some cases, the stool sample or derivative thereof may be directed to the sensor without intermediary vessels. In such cases, the stool sample or derivative thereof may be directed to the sensor (e.g., an inlet of the sensor) via one or more forces, e.g., positive pressure, negative pressure (e.g., aspiration), centrifugal, capillary, gravitational, frictional, electric, magnetic forces. The sensor and electronics unit may be configured to further process and/or analyze the stool sample or derivative thereof, as described elsewhere herein. For example, the sensor and electronics unit may be used to isolate one or more biomarkers or biological molecules (e.g., nucleic acid molecules, proteins, lipids, carbohydrates, metabolites, or a combination thereof) from the stool sample or derivative thereof. In such cases, the sensor and electronics unit may comprise an inlet where reagents (e.g., buffers, functionalized beads) may be introduced. Functionalized beads may be used to isolate the one or more biological molecules. For example, the beads may comprise nucleic acid sequences that may hybridize or anneal to the nucleic acid molecules from the stool sample or derivative thereof. In some cases, the beads comprise aptamers, biotin, antibodies, silica or other moieties useful for capturing other biological molecules, e.g., proteins, nucleic acids. In certain cases, the beads comprising the nucleic acid molecules may be immobilized and/or washed, to remove other particles from the stool sample and increasing the purity of the sample.

The sensor and the electronics unit, and any components thereof, may be part of a single housing, or each of the components may comprise multiple housings that are connected to one another. For instance, the sensor may comprise a first housing that has compartments to include the electronics unit or analysis or processing units, e.g., a microfluidic device. In other instances, the microfluidic device and/or electronics unit may be coupled to the sensor (e.g., electrically and/or fluidically via tubing, a communication interface, wires, etc.).

The sensor may be or comprise a biosensor. The sensor may comprise a microfluidic device or unit (also referred to herein as a “chip”). The microfluidic device may comprise one or more channels. In some instances, the channel may comprise a dimension (e.g., length, width, height) that can be between a range of from about 5 micrometers (μm) to about 10,000 μm. For instance, the channel dimension may be at least about 5 μm, at least about 10 μm, at least about 20 μm, at least about 30 μm, at least about 40 μm, at least about 50 μm, at least about 60 μm, at least about 70 μm, at least about 80 μm, at least about 90 μm, at least about 100 μm, at least about 200 μm, at least about 300 μm, at least about 400 μm, at least about 500 μm, at least about 600 μm, at least about 700 μm, at least about 800 μm, at least about 900 μm, at least about 1,000 μm, at least about 2,000 μm, at least about 3,000 μm, at least about 4,000 μm, at least about 5,000 μm, at least about 6,000 μm, at least about 7,000 μm, at least about 8,000 μm, at least about 9,000 μm, at least about 10,000 μm, or more. The channel dimension may be at most about 10,000 μm, at most about 9,000 μm, at most about 8,000 μm, at most about 7,000 μm, at most about 6,000 μm, at most about 5,000 μm, at most about 4,000 μm, at most about 3,000 μm, at most about 2,000 μm, at most about 1,000 μm, at most about 900 μm, at most about 800 μm, at most about 700 μm, at most about 600 μm, at most about 500 μm, at most about 400 μm, at most about 300 μm, at most about 200 μm, at most about 100 μm, at most about 90 μm, at most about 80 μm, at most about 70 μm, at most about 60 μm, at most about 50 μm, at most about 40 μm, at most about 30 μm, at most about 20 μm, at most about 10 μm, at most about 5 μm, or less. In some instances, the dimension can be between a range of from about 10 μm to about 200 μm. Alternatively, the dimension can be less than about 10 μm. Alternatively, the dimension can be greater than about 200 μm. In some instances, the flow rate of the sample or fluid entering the junction can be between about 5 microliters (μL)/minute (min) and about 10,000 μL/min.

The sensor, the microfluidic device, or both, may comprise a plurality of regions for further processing of the stool sample. Non-limiting examples of processing operations include: filtration, heating, cooling, applying an electric potential, separation of one or more analytes (e.g., via filtration, chromatography, electrokinetics, centrifugation), partitioning (e.g., forming droplets), flow cytometry, merging of reagents, mixing, interfacing with an analysis instrument (e.g., mass spectrometer, sequencing instrument, etc.). The sensor or the microfluidic device may be configured to perform one or more processing operations, in any combination or order.

In some cases, the sensor, the microfluidic device, or both may comprise a plurality of filters. For example, the stool sample or derivative thereof may be introduced into the microfluidic device and undergo filtration. In some cases, the stool sample is subjected to conditions sufficient to filter the sample using gravitational filtration or sedimentation. The gravitational filtration or sedimentation may occur prior to, during, or following introduction of the stool sample or derivative thereof to the microfluidic device. Alternatively or in addition to, the sensor or the microfluidic device may comprise a plurality of filters. A filter of the plurality of filters may comprise a membrane, beads, diaphragms, colloids, weir filters, pillar filters, cross-flow filters, solvent filters, sieves, or any other filter. In some cases, the microfluidic device may comprise geometric features that may be used to filter the stool sample or derivative thereof. The geometric features may be any useful geometry or size for filtration. The geometric features may comprise one or more geometries, e.g., pillars, pyramids, pegs, cylinders, spheres, boxes. The geometric features may comprise one or more sizes. In some cases, the geometric features comprise micropillars that may decrease in size across one or more dimensions (e.g., length, width, height, or a combination thereof) to generate a gradient of size filters. In some cases, the geometric features comprise micropillars that decrease in size across one or more dimensions to generate discrete zones of size filters. The filters may be used to filter out debris or particles larger than a specified threshold. In some cases, the microfluidic device may comprise features that have a dimension of 1 micrometer (m), 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1000 μm. In some cases, the microfluidic device may comprise features that have a dimension of at least 200 nanometers (nm), 300 μm, 400 μm, 500 μm, 600 μm, 700 μm, 800 μm, 900 μm, 1 μm, 2 μm, 3 μm, 4 μm, 5 μm, 6 μm, 7 μm, 8 μm, 9 μm, 10 μm, 20 μm, 30 μm, 40 μm, 50 μm, 60 μm, 70 μm, 80 μm, 90 μm, 100 μm, 200 μm, 300 μm, 400 μm, 500 μm, 600 μm, 700 μm, 800 μm, 900 μm, 1000 μm. In some cases, the microfluidic device may comprise features that have a dimension of at most 1000 μm, 900 μm, 800 μm, 700 μm, 600 μm, 500 μm, 400 μm, 300 μm, 200 μm, 100 μm, 90 μm, 80 μm, 70 μm, 60 μm, 50 μm, 40 μm, 30 μm, 20 μm, 10 μm, 1 μm. It will be appreciated that the microfluidic device may have features in a range of sizes, e.g., 20-100 μm.

In some instances, the stool sample comprises one or more cells. The cell may be a bacterial cell. The cell may be a fungal cell. The cell may be a mammalian cell from a subject (e.g., a human cell). The stool sample may comprise a mixture of cells. In some instances, the stool sample comprises a virus. The stool sample may comprise a mixture of biomarkers or biomolecules from a cell or virus in a sample. For instance, the biomarker or biomolecule may comprise a protein, nucleic acid molecule, lipid, carbohydrate, glycoprotein, lipoprotein, or a variation or combination thereof. One or more biomarkers or biomolecules may be isolated from the sample, as described elsewhere herein.

The sensor may comprise a region for lysing the one or more cells (or viruses). The lysis region may be integrated into a microfluidic device of the sensor and may comprise a plurality of channels and may optionally be integrated with the electronics unit of the sensor. For instance, the sensor may comprise a microfluidic device comprising built-in electronics or electrical connections for performing electrolysis of a cell in the stool sample. In another example, the sensor may comprise a region for lysis, and the lysed product may be delivered to the microfluidic device for further processing. The lysis region (of the sensor or microfluidic device) may comprise a heating element (e.g., heating pad) that may be used to lyse the one or more cells. The heating element may take on any useful configuration—e.g., a fan, a heating pad, coils, and may generate heat conductively, inductively, convectively, etc. In some cases, a polymer (e.g., polyimide) may be used for the heating pad. The heating element may be coupled by a power supply or battery. The heating element may be controlled by a thermistor connected to a relay and/or controller that may be used to set the target temperature and duration of time for the target temperature. For example, for cell lysis, a temperature range of about 75-80 degrees Celsius (C) may be applied to the lysis region for about 30-60 seconds. It will be appreciated that any range of temperature above ambient temperature may be applied to the sensor using the heating element, e.g., at least 30° C., at least 40° C., at least 50° C., at least 60° C., at least 70° C., at least 80° C., at least 90° C., at least 100° C. or more.

Alternatively or in addition to, other methods of lysis may be employed. Lysis of one or more cells may comprise: electrical lysis, ultrasonic lysis (i.e., application of ultrasonic energy), laser or other focused energy lysis, mechanical lysis (e.g., using beads, shearing, agitation, etc.), biochemical lysis (e.g., using detergents, enzymes, etc.), or a combination thereof. In some cases, lysis may be achieved using electrical lysis, and an electric field or current may be applied to the chip to lyse the cells. In such cases, the electric field may be applied through electrodes or electrical inlets configured to integrate with the electronics unit of the sensor, or in the case when the sensor comprises a microfluidic unit, an electronics unit may be coupled to or comprised within a microfluidic device of the sensor. In some cases, the lysis region may comprise winding channels for the application of ultrasonic energy for ultrasonic lysis. In some cases, lysis may be achieved chemically or biologically via addition of a chemical (e.g., detergent, chaotropes) or a biological agent (e.g., enzyme). For example, a detergent that is ionic (e.g., sodium or sarcosyl dodecyl sulfate), and/or a non-ionic detergent (e.g., Triton-X 100, Tween-20, CHAPS) may be used to lyse the cells. The lysis of the one or more cells in the stool sample may yield a lysed stool sample.

In some cases, one or more biological molecules (also referred herein as “biomolecules,” e.g., proteins, nucleic acids, lipids, carbohydrates, glycoproteins, lipoproteins, or a variation or combination thereof) may be purified, isolated, or extracted, and the purified, isolated or extracted product may be analyzed from the stool sample or derivative thereof. The sensor may be used to analyze a nucleic acid molecule from the one or more cells in the stool sample. In some cases, the sensor (e.g., comprising the microfluidic device) comprises a region for isolation and/or purification of a nucleic acid molecule, e.g., DNA, RNA, mitochondrial DNA, etc. The isolation region may comprise one or more inlets, one or more channels, and/or one or more outlets. In some cases, the isolation of the nucleic acid molecule comprises: (i) providing a sample comprising one or more nucleic acid molecules, a plurality of buffers, and air into an inlet of the sensor, (ii) using the beads to bind the one or more nucleic acid molecules from the sample, (iii) releasing the one or more nucleic acid molecules from the beads, and (iv) isolating the one or more nucleic acid molecules. In some cases, the beads may comprise silica magnetic beads. In some cases, the sample comprising one or more nucleic acid molecules may come from the lysed stool sample. In some cases, the plurality of buffers may comprise a binding buffer, a wash buffer, and an elution buffer. The binding buffer may be used to conjugate or couple the nucleic acid molecules from the lysed stool sample to the beads (e.g., via hybridization to random nucleic acid primers attached to the bead). One or more processes may occur in one of the channels of the sensor. For example, the lysed stool sample, binding buffer, beads, wash buffer, and elution buffer, and air may be introduced via one or more inlets into the sensor or portion thereof (e.g., a microfluidic device within the sensor) sequentially or simultaneously. The nucleic acid molecules may be bound or coupled to the beads in the channel and immobilized to a region in the channel (e.g., using a magnet). The remaining particles (e.g., proteins, lipids, carbohydrates, other biomolecules) and/or buffers may be washed out of the chip (e.g., via an outlet), leaving behind a purified product. The elution buffer may then be used to unbind the nucleic acid molecules from the beads and isolate the nucleic acid molecules from the chip via an outlet.

In some cases, a protein or other biological particle (e.g., a cell or cellular components such as nucleic acid molecules) may be purified. In such cases, the method may comprise (i) providing a sample comprising one or more biological particles, a plurality of buffers, and air into an inlet of the biosensor, (ii) using functionalized beads to bind the one or more biological particles from the sample, (iii) releasing the one or more biological particles from the beads, and (iv) isolating the one or more biological particles. In some cases, the beads may be silica magnetic beads. In some cases, the sample comprising one or more biological particles may come from the lysed stool sample. In some cases, the plurality of buffers may comprise a binding buffer, a wash buffer, and an elution buffer. The binding buffer may be used to conjugate or couple the biological particles from the lysed stool sample to the beads (e.g., via antibody-antigen binding). One or more processes may occur in one of the channels of the biosensor. For example, the lysed stool sample, binding buffer, beads, wash buffer, and elution buffer, and air may be introduced via one or more inlets into the sensor (or a microfluidic device) sequentially or simultaneously. The biological particles may be bound or coupled to the beads in the channel and immobilized to a region in the channel (e.g., using a magnet). The remaining particles and/or buffers may be washed out of the chip (e.g., via an outlet), thereby generating a purified product. The elution buffer may then be used to unbind the biological particles from the beads and isolate biological particles from the sensor or chip via an outlet.

A bead, as described herein, may be a solid or semi-solid particle. The bead may be a gel, a solid, a porous solid, etc. The bead may be a polymeric bead comprising one or more polymers. The gel bead may include a polymer matrix (e.g., matrix formed by polymerization or cross-linking) or an interpenetrating network of polymers. One or more polymers of the bead may be randomly arranged, such as in random copolymers, and/or have ordered structures, such as in block copolymers. The bead may comprise a macromolecule or may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be a solid particle (e.g., a metal-based particle including but not limited to iron oxide, gold or silver) covered with a coating comprising one or more polymers. The bead may be a silica particle. The bead may comprise one or more materials, e.g., metal and silica (e.g., glass), ceramic, etc. The bead may be custom-built or commercially available. In some instances, the bead may have a diameter of between 10 nanometers and 10 micrometers. A range of bead sizes may be utilized in each embodiment. Alternatively, the bead may have a diameter below 10 nanometers or above 10 micrometers.

The sensor may comprise or be coupled to a plurality of reservoirs. The reservoirs may house the water and/or buffers. For example, a first reservoir may house the binding buffer, a second reservoir may house the wash buffer, a third reservoir may house the elution buffer, and a fourth housing may house water and/or a cleaning or sterilization agent (e.g., bleach). One or more of the reservoirs may be coupled to the biosensor (i.e., microfluidic device) via tubing, a valve (e.g., pinch valve, diaphragm valve, ball valve), and/or one or more pumps. The valves may control the flow, flow rate, flow volume, etc. of the fluid in the reservoir to the biosensor. The pump may be used to control the flow rate or flow volume of the fluid in the reservoir to the sensor.

Any combination of parts and processes described herein may be used. For example, the sensor (which may comprise a microfluidic device) may comprise a region for gravitational sedimentation, a region for size filtration, a region for cell lysis, and a region for the collection and/or isolation of the biomolecule (e.g., DNA). The sensor may comprise a series of biosensors that are coupled and/or connected in series or in parallel. The biosensors may be fluidically coupled via tubing, vessels, tubes, or any other useful connection method.

In some cases, one or more of the processes described herein are performed automatically. For example, pumping of one or more fluids (e.g., buffers, air) may be automated using a micro-controller that may be coupled to the valves, magnets, and/or pumps. The micro-controller may be any suitable micro-controller, e.g., an Arduino controller, Raspberry Pi controller, etc. In some instances, the electronics unit of the sensor comprises the micro-controller that is programmable. In some instances, the electronics unit of the sensor comprises a printed circuit board (PCB). Automation of one or more processes may be used to streamline operation, minimizing human error and decreasing operation time. For example, a method described herein may be configured to be performed within a 5-10 minute time frame or a different time frame. For instance, a method or process (e.g. sample collection, homogenization, filtering, lysis, processing, isolation or extraction of a biomolecule, storage, incubation, reaction durations, etc.) described herein may be performed in at most 10 seconds, at most 20 seconds, at most 30 seconds, at most 40 seconds, at most 50 seconds, at most 60 seconds, at most 70 seconds, at most 80 seconds, at most 90 seconds, at most 100 seconds, at most 5 minutes, at most 10 minutes, at most 20 minutes, at most 30 minutes, at most 1 hour, at most 2 hours, at most 3 hours, at most 4 hours, at most 5 hours, or more. It will be appreciated that the methods and processes described herein may be performed in a range of time frames, e.g., between 30 seconds and 30 minutes, between 10 minutes and 90 minutes, etc.

The sensor may have a variety of useful functions and/or features. The sensor, or a portion thereof, may be configured to be disposable. In some cases the sensor, or component thereof, may comprise recyclable or disposable plastics or polymers, e.g., polyethylene terephthalate, polyethylene, polyvinyl chloride, polypropylene, polystyrene, acrylic, polycarbonate, polylactic acid, nylon, or a derivative thereof (e.g., high density, low density, cross-linked, etc.). In some cases, the sensor or component thereof may comprise a plurality of polymers. The polymer may be a biopolymer or a synthetic polymer. The polymer may be crosslinked. The polymer may be a co-polymer, e.g., block copolymer, homopolymer, heteropolymer, etc. In some cases, the disposable component of the sensor may be replaced for a new sample to be processed. The replacement of the disposable component of the sensor may be performed manually or may be automated. In some cases, the sensor, or component thereof, may comprise a mechanism to eject used chips and load new chips.

The sensor may be used to analyze a parameter of the processed stool sample. The parameter may be a health, physiological or pathological parameter, or may be a parameter indicative of or a proxy for a health, physiological, or pathological condition. In some cases, the parameter is the presence or concentration of a biological material. The biological material may be a cell or a component of a cell, e.g., a nucleic acid, a protein, a peptide, a polypeptide, a lipid, a carbohydrate, an organelle, a macromolecule, biomolecule, metabolite, or a derivative or combination thereof. The biological material may be from the subject or may be from a virus, a microbe (e.g., bacteria, protist, fungus) or other organism. The biological material may comprise a bodily fluid or component thereof, e.g., blood, serum, plasma, sputum, semen, saliva, urine, feces, cerebrospinal fluid, synovial fluid, tears, mucus, lymphatic fluid, bile, pus, etc. In some cases, the stool is analyzed for the presence or concentration of a non-biological particle. Non-limiting examples of non-biological particles include organic compounds, non-organic compounds, small molecules (e.g., drugs or compounds), metals, minerals, etc. In some cases, the chemical or biological material or non-biological particle may be indicative of a health, physiological, or pathological condition. For example, the biological material may be a nucleic acid sequence (e.g., DNA, RNA) indicative of a disease or a predisposition for a disease. The presence of the biological material may indicate the presence of a virus or organism (bacteria, fungi, protist, etc.). In another example, the biological material may be a protein which may be indicative of a disease or a predisposition for a disease.

Analyzing the parameter of the stool sample or derivative thereof (e.g., processed stool sample) may comprise one or more biochemical techniques and/or assays. Analysis of the sample may comprise imaging and/or microscopy (e.g., fluorescence microscopy, bright-field or transmitted light microscopy, dark field microscopy, polarized light microscopy, differential interference contrast microscopy, phase contrast microscopy, etc.). Alternatively or in addition to, analysis of the stool sample or derivative thereof may comprise one or more biochemical assays. For example, the analysis may comprise nucleic acid analysis, such as sequencing, PCR, in situ hybridization or another nucleic acid analysis technique. The analysis may comprise protein or peptide analysis, e.g., mass spectrometry, nuclear magnetic resonance, Raman spectroscopy, Fourier-transform infrared spectroscopy, X-ray photoelectron spectroscopy, absorption spectroscopy, emission spectroscopy, elastic scattering spectroscopy, emission spectroscopy, impedance spectroscopy, resonance spectroscopy, quartz crystal microbalance with dissipation, immunoassays (e.g., Western blot, immunocytochemistry or immunohistochemistry), crystallography or other protein analysis method. A combination of techniques may be used. For example, the sample may be imaged and subjected to a biochemical assay (e.g., sequencing, PCR, immunoassays, etc.)

At any convenient or useful operation, the sensor or a portion thereof (e.g., a microfluidic device) may be stored for later use. For instance, it may be useful to store the sensor or portion thereof (e.g., chips) prior to, during, or following sample processing and/or analysis. In some cases, the sensor may be stored for further analysis. Storage may occur during any convenient process, e.g., after introduction of the stool sample or derivative thereof to the sensor, after filtration, after lysis, after purification, etc. In some cases, the storage of the sensor or chip may occur following analysis of the stool sample or derivative thereof, and the chip may be stored and subsequently re-analyzed. In some cases, the sensor or chip may comprise a plurality of reagents and may be stored (e.g., available “off-the-shelf”) prior to use. The sensors may be configured to be activated or usable following storage, e.g., via heating or cooling, resuspension in a solution or buffer, etc. The storage of the sensors or chips may comprise drying, chilling, freezing, fermenting, curing, lyophilizing, heating, pasteurizing, or adding preservatives or stabilization reagents to the chip. Non-limiting examples of preservatives or stabilization reagents include: formaldehyde, paraformaldehyde, tert-butylhydroquinone, antimicrobial agents, e.g., sorbic acid, sodium sorbate, benzoic acid, sodium benzoate, hydroxybenzoate, sulfur dioxide, sulfites, nitrite, nitrate, lactic acid, propionic acid, isothiazolinones, phenol derivatives, ascorbic acid, butylated hydroxytoluene, gallic acid, tocopherols, etc.

In some cases, one or more reagents and/or the purified product (e.g., isolated biological particle) may be stored. In some cases, the purified product (e.g., isolated DNA) may be stored for further analysis. In some cases, the storage of the purified product may occur prior to or following analysis of the purified product. Storage of the purified product may comprise drying, chilling, freezing, fermenting, curing, lyophilizing, heating, pasteurizing, or adding preservatives or stabilization reagents e.g., EDTA, TE buffer, formaldehyde, paraformaldehyde, tert-butylhydroquinone, antimicrobial agents, e.g., sorbic acid, sodium sorbate, benzoic acid, sodium benzoate, hydroxybenzoate, sulfur dioxide, sulfites, nitrite, nitrate, lactic acid, propionic acid, isothiazolinones, phenol derivatives, ascorbic acid, butylated hydroxytoluene, gallic acid, tocopherols, etc.

The sensor or the electronics unit of the sensor may comprise a communication interface that allows for transmitting and/or receiving data corresponding to the parameter of the stool sample or of the stool collector (e.g., status of collection or processing, errors in the stool collector or sample collection process, etc.). In some cases, the data may be transmitted to an electronic device (e.g., via the electronics unit) in communication with the communication interface. The communication interface may be a wireless communication interface, e.g., a Wi-Fi interface, a near-field communication interface, or a Bluetooth interface. The electronic device may be a device that may communicate with the communication interface. The electronic device may be a mobile device (e.g., a smart phone, tablet, laptop, etc.). Alternatively or in addition to, the communication interface may be a wired communication interface. The sensor may comprise a port for communication and/or a power supply (e.g., universal serial bus (USB), USB-type C, Thunderbolt, Ethernet, etc.).

The electronics unit of the sensor may comprise one or more electronics. The electronics unit may comprise a microcontroller, a detection system, a sensor, a communication interface, or a combination thereof. An electronics unit may be integrated with a plurality of sensors or may be coupled to the one or more sensors. The electronics may be used for information processing, signal processing, and may comprise any suitable part including, but not limited to: vacuum tubes, transistors, diodes, integrated circuits, electrical components, optoelectronics, wires, motors, generators, batteries, switches, relays, transformers, resistors, transmitters, receivers. The electronics may comprise analog circuits, digital circuits, or a combination thereof. The electronics unit may comprise units that are configured to integrate with other analysis units, e.g., sequencing units, mass spectrometer, FTIR, etc. The electronics unit may comprise electrodes, electrical inlets, or electrical units that comprise or are configured to couple to electrodes, which may be used in one or more processing operations (e.g., cell lysis) on the sensor or portion thereof.

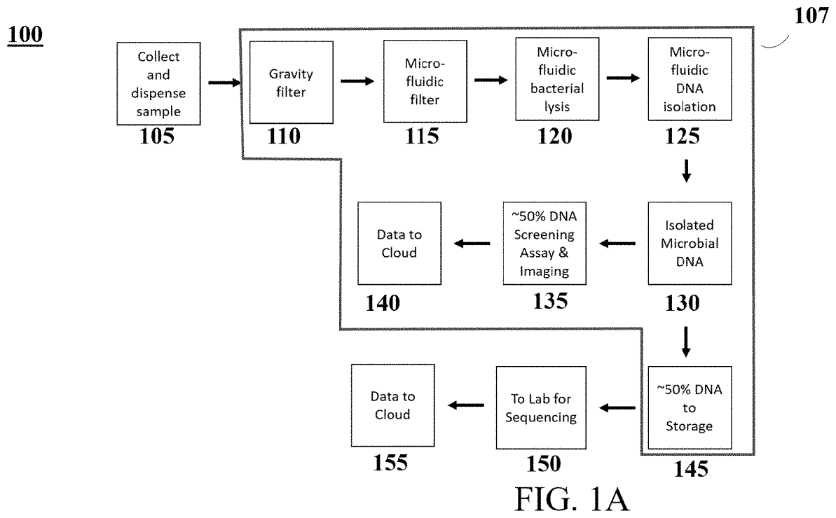

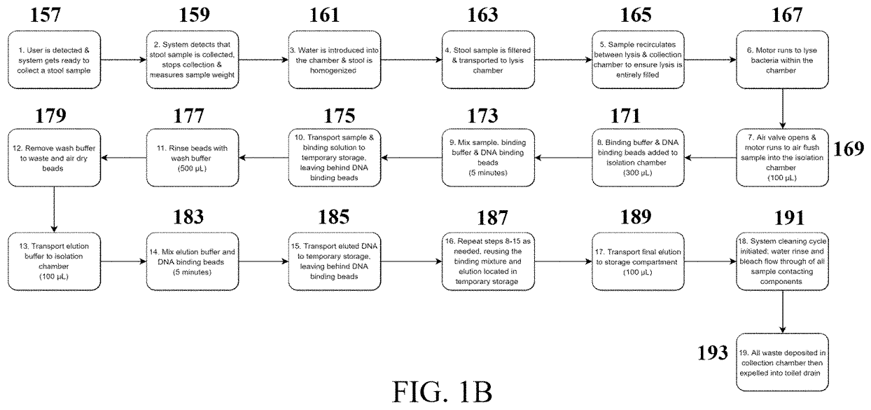

FIG. 1A schematically illustrates an example workflow 100 for analyzing a stool sample. In process 105, a stool sample may be collected using a stool collector, which may be coupled to a toilet. Process 105 may comprise sample processing to generate a derivative of a stool sample, e.g., a homogenized stool sample, which may be collected and/or directed to a sensor 107. In process 110, the stool sample or derivative thereof is subjected to further processing. In some cases, the processing comprises filtration via sedimentation, which may filter out larger particles, debris, etc. The gravity filtration or sedimentation may occur in a component of the sensor (e.g., a microfluidic device). In process 115, the stool sample may be further processed in the sensor. In some cases, the processing comprises filtration in a microfluidic device. The microfluidic device may comprise a plurality of pillars and channel heights that allow for sequential filtering of particles by size. In process 120, the filtered sample may be subjected to conditions sufficient for lysis of one or more cells in the sample. For example, the sample may be heated, or introduction of lysis reagents may be introduced. In process 125, purification may be performed. In some cases, purification comprises isolation of one or more biological particles from the stool sample. The biological particle may be a constituent of the cell, (e.g., a nucleic acid molecule, a protein, a peptide, a lipid, a carbohydrate, or a combination thereof). In some cases, nucleic acids (e.g., DNA) from the cell or cells may be isolated. Isolation of the nucleic acid molecules may occur using a plurality of beads. In some cases, the beads are used to couple the nucleic acid molecules from the cell or cells. The beads may be magnetic, and application of a magnetic force may immobilize the beads. The beads may then be washed, and waste products (i.e., not coupled to the beads) may be eluted out of the chip. In process 130, the biological particles (e.g., nucleic acid molecules) may be isolated, e.g., using beads. In process 135, a portion of the isolated biological particles may be further processed and/or analyzed. For example, the nucleic acids may be subjected to a hybridization assay and imaging or microscopy. In such cases, fluorescent molecules may be introduced to detect the presence of a nucleic acid sequence. In process 140, the data from process 135 may be transmitted via a communication interface to a cloud storage and/or computing system. A portion of the isolated biological particles from 130 may also be stored in process 145. For example, the isolated biological particles may be frozen or dried. In process 150, the isolated portion of biological particles from 130 may further be sent to an offsite location for further processing and analysis. For example, 150 may comprise nucleic acid sequencing, PCR, Southern blot or other nucleic acid analysis. In some cases, the biological particle is a protein and process 150 may comprise a Western blot, immunoassay, or other protein assay. In process 1055, data obtained from process 150 may be transmitted via a communication interface to cloud storage and/or a computing system. Processes 110, 115, 120, 125, 130, 135, 140, and 145, or a combination thereof, may occur in the sensor 107 or a portion thereof.

FIG. 1B shows schematically another example workflow for automatically processing a stool sample according to some embodiments described herein. The workflow may be performed using a stool collector and collection unit (which may, in some instances, be referred to as a “system”). In process 157, the stool collector may detect the user or subject and prepare for collection of the stool sample. In process 159, the system (e.g., stool collector) may detect that the stool sample has been collected and may stop collection. The stool collector may comprise, for instance, a sensor (e.g., a weight sensor) to determine that stool sample collection is sufficient or finished. In process 161, water or other homogenizing fluid may be introduced to homogenize the stool sample, which may occur in a chamber or other vessel or container. In process 163, the stool sample may be filtered and transported a lysis unit (e.g., lysis chamber). In process 165, the sample may be recirculated between the lysis and collection chamber to ensure that the lysis unit is sufficiently filled. In process 167, the stool sample or derivative thereof may be further processed, e.g., via lysis. The lysis may comprise mechanical lysis (e.g., using a motor and an impeller, as described elsewhere herein). In process 169, air or other gas or fluid may be introduced (e.g., via a valve) into an isolation unit, which may be in fluidic communication with the lysis unit or may be performed in the lysis unit. In process 171, a binding buffer and beads (e.g., comprising capture molecules, such as nucleic acid molecules, proteins, antibodies, etc. that are configured to bind to a biomolecule) may be introduced into the isolation unit. In process 173, the stool sample or derivative thereof may be mixed with the beads and binding buffer. The bead-sample mixture may be transported to a storage unit in process 175. Further processing may be performed in process 177, such as rinse the beads with wash buffer. The wash buffer may be removed and the beads may optionally be dried in process 179. In process 181, an elution buffer may be transported to the isolation unit. The elution buffer may be mixed with the beads in process 183. In process 185, the eluted (or otherwise extracted or isolated) biomolecule may be transported to another storage unit, which may be the same or different as the storage unit in process 175. In process 187, any number of operations may be repeated (e.g., 171, 173, 175, 177, 179, 181, 183, and 185). In process 189, the final eluent (or isolated or extracted biomolecule) may be transported to another storage unit (which may be the same or different as those in process 175 or 185. In process 191, the system may be cleaned, e.g., using water, bleach, etc. In process 193, waste may be deposited in a waste unit, or in the collection or homogenization chamber and expelled into the toilet drain. As described herein, any combination or all of the processes described herein may be performed automatically.

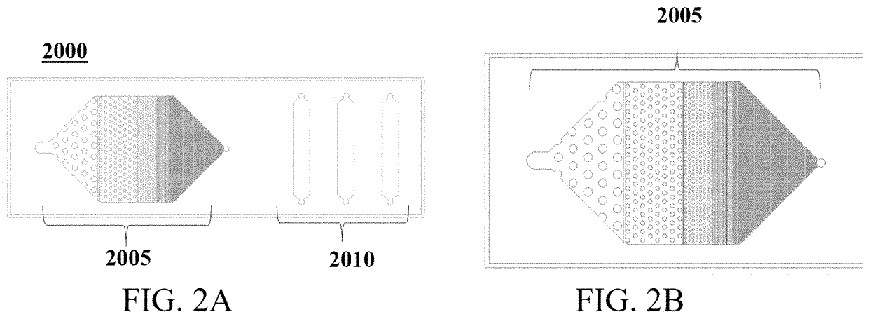

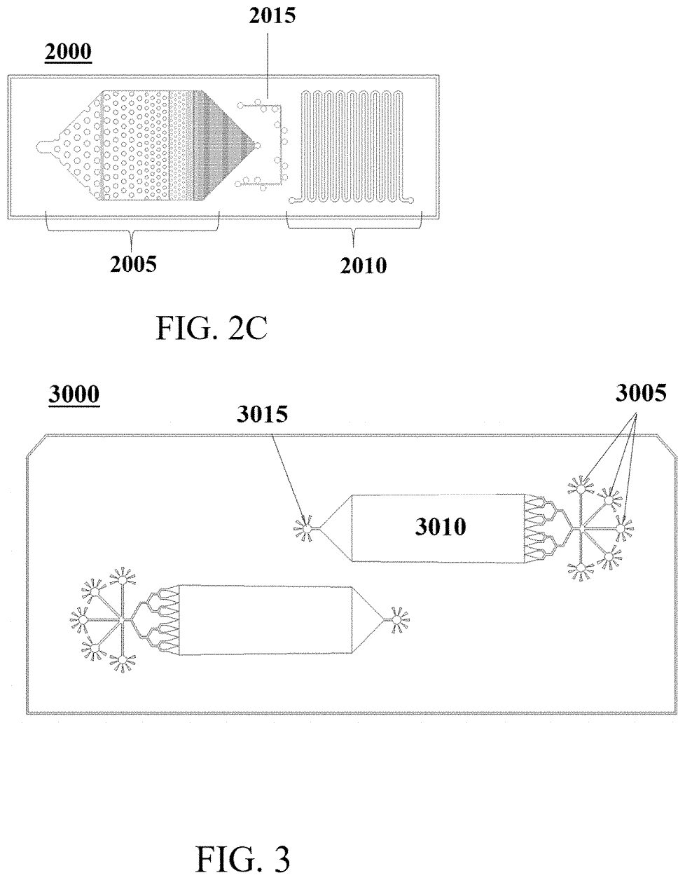

FIG. 2 shows a schematic of a portion of a sensor to analyze a parameter of the stool sample or derivative thereof (e.g., processed stool sample). The sensor may comprise a microfluidic device 2000, as shown in FIG. 2A. Region 2005 of the microfluidic device 2000 may comprise a plurality of features (e.g., micropillars) of varying size (see FIG. 2B), which may be used to filter the sample by size. Region 2005 may comprise a plurality of regions or zones with micropillars of a defined size. For example, the largest micropillars may filter particles of about 1 mm, which may pass through subsequently smaller micropillars. The height of the microchannel may also decrease with the feature size. The smallest features may filter particles of about 50 micrometers. The feature sizes, channel dimensions, and other dimensions of the sensor may be tuned. For example, the sensor may comprise devices across length scales, e.g., nanometers, millimeters, centimeters, etc. Region 2010 may be coupled fluidically to region 2005. Region 2010 may comprise a plurality of lysis channels. The filtered stool sample from region 2005, which may comprise one or more cells, may be transferred, e.g., via tubing, to region 2010. Lysis may be performed in region 2010. In some cases, a heating pad (e.g., polyimide heating pad) may be placed adjacent to the sensor to heat the sample, thereby lysing the one or more cells. The heating pad may be powered by a battery or coupled to an electrical outlet or power supply. In some cases, the heating pad is controlled using a thermistor that may be embedded in the sensor and connected to a relay or controller (not shown). Alternatively or in addition to, lysis may be performed using other methods, including but not limited, applying an electric current or field, ultrasonic energy, laser or focused energy, or via mechanical lysis (e.g., using beads, shearing or agitation), chemical lysis (e.g., using surfactants, detergents, enzymes, etc.), or a combination thereof. Following lysis, the filtered, lysed stool sample may be retrieved from the sensor via an outlet. FIG. 2C shows a schematic of a microfluidic device of a sensor to analyze a parameter of the stool sample comprising a filter region 2005, and a lysis region 2010 comprising a winding channel that may be used for the application of ultrasonic energy over time. The microfluidic device may additionally comprise a channel 2015 which may comprise electrical inlets for applying an electric field or electric current for cell lysis. A portion of region 2005 may be connected to the lysis region 2010 (e.g., using tubing). The stool sample may be filtered in region 2005, e.g., to remove debris, and the filtered stool sample may be input into the lysis region 2010. The sample may be lysed, as described herein, and the product may be retrieved from the microfluidic device or the sensor.

The filtered, lysed stool sample may be coupled to a second microfluidic device for further processing, e.g., purification. FIG. 3 schematically shows an example microfluidic device used for further processing of the stool sample. The microfluidic device 3000 may be coupled to one or more of the microfluidic devices 2000 shown in FIG. 2. In some cases, the microfluidic device 3000 may be a part of microfluidic device 2000 shown in FIG. 2. Microfluidic device 3000 may comprise inlet ports 3005, which may be coupled to one or more reservoirs (not shown). In some cases, each port 3005 may be connected to a separate reservoir. In some cases, one or more ports 3005 may be connected to the same reservoir. The reservoir or reservoirs may comprise any useful fluid to be introduced into the microfluidic device 3000. For example, a reservoir may comprise air, water, buffers, or other useful reagents. In some cases, buffers may be used for purification of the sample (e.g., isolation of a biological particle such as a nucleic acid molecule). In such cases, the reservoir or reservoirs may comprise air, a binding buffer, a wash buffer, and an elution buffer. A plurality of beads (e.g., magnetic beads) may be introduced via a separate port or may be mixed in with one of the buffers (e.g., binding buffer). Each reservoir may be fluidically coupled to the microfluidic device 3000. In some cases, the coupling further comprises one or more valves, (e.g., pinch valves), which may be used to control the flow rate of the fluid in each reservoir. One or more ports 3005 may be coupled (e.g., fluidically) to the microfluidic device 2000 shown in FIG. 2. For example, following filtration and lysis of the stool sample, the filtered, lysed product may exit through an outlet and enter through an inlet port 3005 into the second microfluidic device. In some instances, the microfluidic device 3000 may be integrated (i.e., not removable) from the microfluidic device 2000 of FIG. 2.

The microfluidic device 3000 may also comprise a channel 3010 for sample processing. In some cases, the filtered, lysed stool sample is introduced to the channel 3010 of sensor 3000 via an inlet port 3005. Channel 3010 may also serve as a reaction chamber. Prior to, during or following introduction of the filtered, lysed stool sample to channel 3010, a binding buffer may be introduced via an inlet port 3005, which may be the same or different as the port used to introduce the filtered, lysed stool sample. In some cases, the binding buffer may be co-introduced with a plurality of beads (e.g., magnetic beads). In some cases, the beads may be mixed in with the binding buffer and introduced into channel 3010. The beads may comprise aptamers, biotin, avidin or streptavidin, antibodies, silica, or other moieties useful for capturing biological molecules. For example, the beads may comprise a nucleic acid capture sequence that can couple to or hybridize with nucleic acid molecules in the filtered, lysed stool sample. The beads may be incubated with and coupled to a biological particle (e.g., nucleic acid molecule) from the lysed stool sample. In some cases, the binding buffer comprises reagents to facilitate the binding of the biological particles to the beads. Prior to, during, or following the binding of the beads to the biological particles, a magnet or electromagnetic force may be applied to a portion of the microfluidic device 3000 (e.g., channel 3010). Application of the magnet or electromagnetic force may be used to mobilize or immobilize the beads. The magnets or electromagnetic force may be used to agitate the beads and/or immobilize the beads to a region of the microfluidic device 3000. Following immobilization, the beads, a portion of which may be coupled to one or more biological particles of interest (e.g., DNA), may be washed by introducing a wash buffer from an inlet port 3005. Waste product may be directed out of the microfluidic device 3000 via an outlet 3015. The outlet 3015 may be fluidically coupled to a valve (e.g., pinch valve), which may be coupled to a waste receptacle, a collection unit, a processing unit, and/or a screening unit (not shown). The wash buffer may be used to wash the beads, and the waste products may be directed out of the microfluidic device 3000 via an outlet 3015 to a waste stream or receptacle. The wash step may be repeated any number of useful times. Following washing, the biological particles coupled to the beads may be eluted using an elution buffer, which may be introduced from an inlet port 3005. The elution buffer may remove the captured biological particles (e.g., DNA) from the beads and may be directed to the outlet 3015 of the microfluidic device 3000. The captured biological particles may then be collected. Alternatively or in addition to, the captured biological particles may be directed to a screening assay and/or a collection and storage unit. In some cases, air is introduced via an inlet port 3005 to direct the purified product (e.g., biological particles) to the outlet 3015.

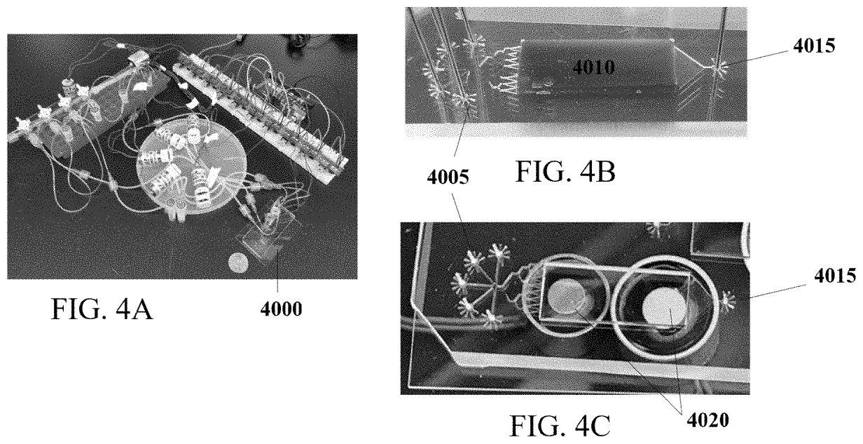

FIG. 4 provides images of a microfluidic device used for analyzing a stool sample. FIG. 4A shows the microfluidic device 4000, which may be substantially similar to the microfluidic device 3000, coupled to a plurality of reservoirs. FIG. 4B shows the microfluidic device 4000 comprising a channel 4010 for processing and/or analyzing a stool sample. The filtered, lysed stool sample may be introduced to the channel 4010, which may be substantially similar to 3010 via an inlet port 4005 which may be substantially similar to 3005. Channel 4010 may also serve as a reaction chamber. Prior to, during or following introduction of the filtered, lysed stool sample to channel 4010, a binding buffer may be introduced via an inlet port 4005, which may be the same or different as the port used to introduce the filtered, lysed stool sample. In some cases, the binding buffer may be co-introduced with a plurality of beads (e.g., magnetic beads). In some cases, the beads may be mixed in with the binding buffer and introduced into channel 4010. The beads may comprise aptamers, biotin, avidin or streptavidin, antibodies, silica, or other moieties useful for capturing biological molecules. For example, the beads may comprise a nucleic acid capture sequence that can couple to or hybridize with nucleic acid molecules in the filtered, lysed stool sample. The beads may be incubated with and coupled to a biological particle (e.g., nucleic acid molecule) from the lysed stool sample. In some cases, the binding buffer comprises reagents to facilitate the binding of the biological particles to the beads.

FIG. 4C shows an image of the microfluidic device 4000 with applied magnets 4020. Prior to, during, or following the binding of the beads to the biological particles, a magnet 4020 or electromagnetic force may be applied to a portion of the microfluidic device 4000 (e.g., channel 4010). Application of the magnet 4020 or electromagnetic force may be used to mobilize or immobilize the beads. The magnets 4020 or electromagnetic force may be used to agitate the beads and/or immobilize the beads to a region of the microfluidic device 4000. Following immobilization, the beads, a portion of which may be coupled to one or more biological particles of interest (e.g., DNA), may be washed by introducing a wash buffer from an inlet port 4005. Waste product may be directed out of the microfluidic device 4000 via an outlet 4015. The outlet 4015 may be fluidically coupled to a valve (e.g., pinch valve), which may be coupled to a waste receptacle, a collection unit, a processing unit, and/or a screening unit (not shown). The wash buffer may be used to wash the beads, and the waste products may be directed out of the device via an outlet 4015 to a waste stream or receptacle. The wash step may be repeated any number of useful times. Following washing, the biological particles coupled to the beads may be eluted using an elution buffer, which may be introduced from an inlet port 4005. The elution buffer may remove the captured biological particles (e.g., DNA) from the beads and may be directed to the outlet 4015 of the microfluidic device 4000. The released biological particles may then be collected. Alternatively or in addition to, the biological particles may be directed to a screening assay and/or a collection and storage unit. In certain cases, the biological particle may remain attached to the bead. In some cases, air is introduced via an inlet port 4005 to direct the purified product (e.g., biological particles) and/or beads to the outlet 4015.

One or more of the processes described herein may be performed automatically. For example, pumping of one or more fluids (e.g., buffers, air) may be automated using a micro-controller that may be coupled to the valves, magnets, and/or pumps. The micro-controller may be any suitable micro-controller, e.g., an Arduino controller, Raspberry Pi controller, etc. The microcontroller may be connected to a power supply (e.g., 12V battery) or other electrical source.

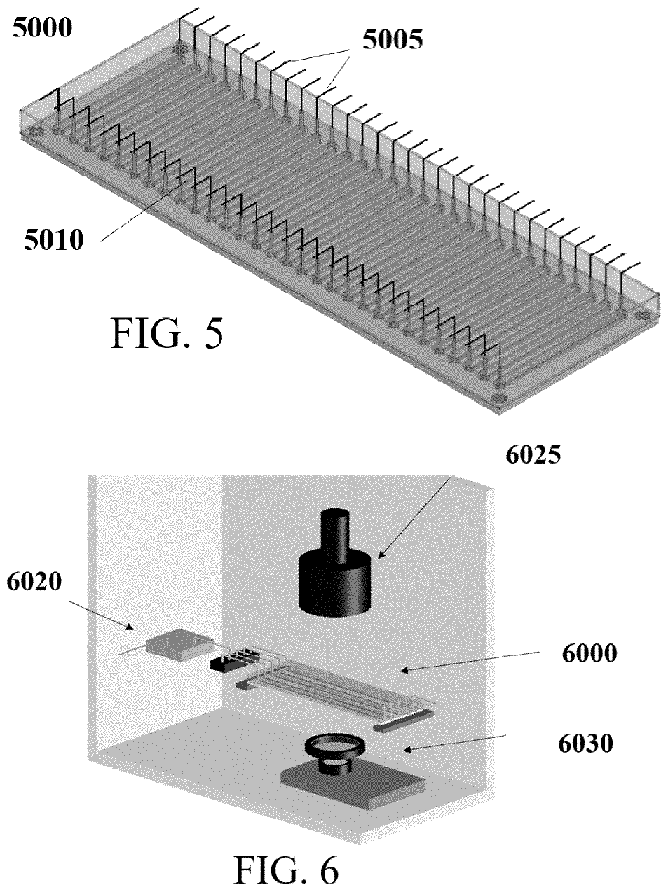

The purified product may be directed to a third microfluidic device for further processing and/or analysis. FIG. 5 schematically shows an example microfluidic device used for analyzing a parameter of the stool sample. The microfluidic device 5000 may be coupled to one or more of the microfluidic device 2000 and 3000 shown in FIGS. 2-3. In some cases, either or both of the microfluidic devices 2000 and 3000 shown in FIGS. 2-3 may comprise or be coupled to the microfluidic device 5000. The microfluidic device 5000 may comprise a plurality of inlet ports 5005 connected to a plurality of channels. In some cases, each port 5005 may be connected to a separate reservoir. In some cases, one or more ports 5005 may be connected to the same reservoir. One or more reservoirs may comprise the purified product (e.g., output from the microfluidic device 3000), and any useful fluid to be introduced into the microfluidic device 5000. For example, a reservoir may comprise reagents used for a screening assay. The reagents may be provided for analyzing a parameter of the purified product. In some cases, the parameter comprises the presence, absence, or quantity of a nucleic acid molecule. In such cases, reagents such as fluorescently labeled nucleotides or nucleic acid molecules (e.g., primers) may be provided, which may hybridize with a portion of the purified product, e.g., via sequence complementarity. Hybridization of the labeled molecule to the purified product may produce a signal (e.g., fluorescence) which may be detected using one or more detectors.

FIG. 6 schematically shows an example system for analyzing a parameter of the processed stool sample. The system or sensor may comprise the microfluidic device 6000, which may be substantially similar to 5000 of FIG. 5, a reservoir 6020, a detector 6030, and an illumination or energy source 6025. The microfluidic device 6000 may comprise a plurality of channels. The microfluidic device 6000 may be coupled to reservoir 6020, which may comprise the sample (e.g., purified product), and/or reagents used for the screening assay. For example, the reservoir may comprise one or more labeled agents that may be used to identify, locate, quantify, or analyze a biological particle of interest. The labeled agents may be nucleic acid molecules (e.g., a primer or barcode sequence), aptamers, proteins, binding molecules (e.g., biotin, avidin, streptavidin), antibodies, or may comprise a useful dye or stains. One or more reagents may be used in the screening assay. For example, one or more labeled nucleic acid primers may be used to identify, label, quantify, or analyze one or more nucleic acid sequence of interest. Alternatively or in addition to, one or more antibodies may be used to bind, identify, label, or analyze a protein or peptide of interest. Antibodies may be labeled primary antibodies or labeled secondary antibody configured to bind to a primary antibody that may bind to the biological particle.

Any useful labeling agent may be used. In some cases, the labeling agent may comprise a fluorophore, a small molecule, a radioisotope, an enzyme, an aptamer, a biotin molecule, etc. In some cases, the labeling agent is configured to generate a signal upon application of a reagent or stimulus. For example, the label may comprise an enzyme (e.g., horseradish peroxidase) that generates a chemiluminescent signal upon the addition of a substrate. In some cases, the labeling agent is configured to generate a signal upon binding of the label or labeled agent to the molecule or biological particle of interest.

The microfluidic device 6000 may be integrated with an illumination or energy source 6025 and/or a detector 6030, which may be integrated with the sensing unit or connected to the sensing unit. The detector 6030 may be capable of detecting a signal, including a signal indicative of the presence or absence of one or more biological particles (e.g., a nucleic acid sequence of interest). The detector may detect multiple signals. The signal or multiple signals may be detected in real-time prior to, during, or following the screening assay. In some cases, a detector can include optical and/or electronic components that can detect signals. Non-limiting examples of detection methods include optical detection, spectroscopic detection, electrostatic detection, electrochemical detection, acoustic detection, magnetic detection, and the like. Optical detection methods include, but are not limited to, light absorption, ultraviolet-visible (UV-vis) light absorption, infrared light absorption, light scattering, Rayleigh scattering, Raman scattering, surface-enhanced Raman scattering, Mie scattering, fluorescence, luminescence, and phosphorescence. Spectroscopic detection methods include, but are not limited to, mass spectrometry, nuclear magnetic resonance (NMR) spectroscopy, and infrared spectroscopy. Electrostatic detection methods include, but are not limited to, gel-based techniques, such as, for example, gel electrophoresis, isoelectric focusing, and isotachophoresis. Electrochemical detection methods include, but are not limited to, electrochemical detection of the biological particle after high-performance liquid chromatography. The detector may comprise microscopy components, e.g., a camera, a photomultiplier tube, pinholes, lenses, dichroic mirrors, etc. Accordingly, the illumination or energy source may comprise emitted light, electromagnetic radiation, acoustic energy (e.g., radiated pressure waves), and/or particles.

In another aspect, disclosed herein are systems and methods for processing a stool sample of a subject. The method may comprise: (a) providing (i) a stool collector coupled to a toilet and (ii) a collection unit coupled to the stool collector; (b) using the stool collector to collect the stool sample of the subject, and (c) in the collection unit, extracting or isolating a biomolecule from the stool sample or a derivative thereof.

In yet another aspect, disclosed herein are systems and methods for processing a stool sample, comprising (a) receiving the stool sample at a first location, in which the stool sample is collected at a second location different than the first location, and wherein the second location is within 1 mile of the first location.

As described herein, the stool collector may comprise any useful geometry for collecting the stool sample, and the stool collector may be coupled to a number of parts, such as the toilet or the collection unit. The collection unit may comprise a sensing unit, which may comprise a sensor and an electronics unit. The electronics unit of the sensor or sensing unit may comprise one or more electronics. The electronics unit may comprise a microcontroller, a detection system, a sensor, a communication interface, or a combination thereof. An electronics unit may be integrated with a plurality of sensors or may be coupled to the one or more sensors. The electronics may be used for information processing, signal processing, and may comprise any suitable part including, but not limited to: vacuum tubes, transistors, diodes, integrated circuits, electrical components, optoelectronics, wires, motors, generators, batteries, switches, relays, transformers, resistors, transmitters, receivers. The electronics may comprise analog circuits, digital circuits, or a combination thereof. The electronics unit may comprise units that are configured to integrate with other analysis units, e.g., sequencing units, mass spectrometer, FTIR, etc. The electronics unit may comprise electrodes, electrical inlets, or electrical units that comprise or are configured to couple to electrodes, which may be used in one or more processing operations (e.g., cell lysis) on the sensor or portion thereof.

The stool collector may be removably coupled to the toilet, non-removably coupled to the toilet, or be a part of the toilet. Similarly, the stool collector may be removably coupled to the collection unit, non-removably coupled to the collection unit, or be a part of the collection unit. For instance, the stool collector may comprise a first housing, which is coupled to the toilet, and the collection unit may comprise a second housing, which second housing is coupled to or configured to couple to the first housing of the stool collector. In certain instances, the stool collector is detachable or removable from the toilet and/or the collection unit. For instance, the stool collector may be coupled to the toilet via one or more fastening mechanisms (e.g., via an interference fit, force fit, shrink fit, location fit, etc. Other fastening mechanisms are possible, such as form-fitting pairs, hooks and loops, latches, threads, screws, staples, clips, clamps, prongs, rings, brads, rubber bands, rivets, grommets, pins, ties, snaps, Velcro, vacuum, seals, or a combination thereof. Alternatively or in addition to, the stool collector may be coupled to the collection unit via one or more fastening mechanisms.

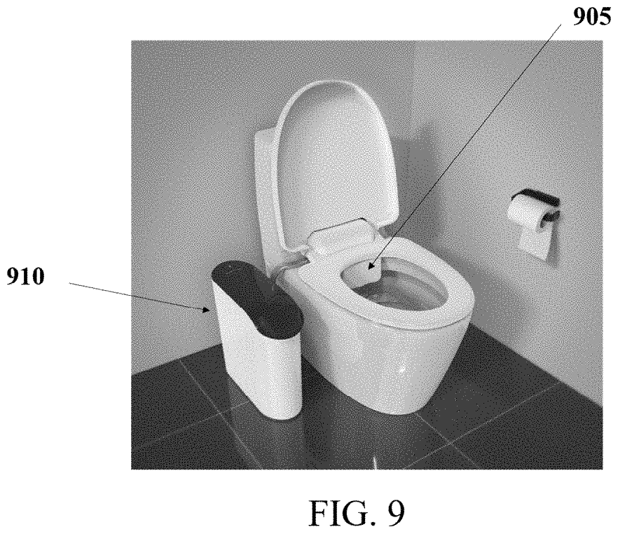

In some instances, the collection unit may be connected to the stool collector and/or toilet via an external part. For instance, the stool collector may comprise a first housing, which is coupled to the toilet, and the collection unit may comprise a second housing, which second housing is coupled to or configured to couple to, via the external part, the first housing. The collection unit may be connected to the stool collector via a fluid flow path (see, e.g., FIG. 9). The part or fluid flow path may comprise any useful connector such as tubing, vessels (e.g., tubes, containers, vials, flasks), valves, clamps, clips, funnels, etc. In some cases, the stool sample or derivative thereof (e.g., homogenized sample) may be directed to the collection unit via tubing and one or more pumps. For example, following the collection of the stool sample or derivative thereof (e.g., homogenized stool sample), the stool sample or derivative thereof may be directed to the collection unit, which may be coupled to the stool collector via tubing and a pump. In such cases, the stool sample or derivative thereof may be pumped through the tubing to the collection unit. The stool sample may be directed to the collection unit via one or more forces, e.g., positive pressure, negative pressure (e.g., aspiration), centrifugal, capillary, gravitational, frictional, electric, magnetic forces. In some cases, the stool sample or derivative thereof may be directed to the collection unit without intermediary vessels. In such cases, the stool sample or derivative thereof may be directed to the collection unit via one or more forces, e.g., positive pressure, negative pressure (e.g., aspiration), centrifugal, capillary, gravitational, frictional, electric, magnetic forces.

In some instances, the collection unit is separate from the stool collector or removably coupled to the stool collector. For instance, the collection unit may be connected to the stool via removable tubing or pipes, removably coupled housings, or the like, such that following collection, the stool sample is deposited or otherwise directed (e.g., via a fluid flow path) to the collection unit, where further processing may be performed, either at the site of collection or elsewhere (e.g., at a clinic or laboratory). In other instances, the collection unit may be provided alone, separate from the stool collector. In such instances, a stool sample (which may be provided separately) may be deposited or directed to the collection unit, which can be used for further processing.

The collection unit may be configured to perform one or more operations for sample processing. The processing can include, in non-limiting examples, sample homogenization, filtration, lysis of cells within a sample, isolation or extraction of a biomolecule from the sample, or a combination of such processes. For instance, after the stool sample is deposited or directed to the collection unit, the stool sample may be homogenized. Accordingly, the collection unit may comprise a homogenization chamber or any necessary apparatuses, reagents, etc. (e.g., fluid inlets, valves, blades, beads, etc.) or access thereto, to homogenize the sample, as described herein. In some instances, the sample may be pre-homogenized (e.g., via the stool collector) and the homogenized sample may be deposited or directed to the collection unit for further processing.

The collection may be configured to perform filtration of the stool sample or derivative thereof. For instance, the stool sample or homogenized stool sample may be subjected to conditions sufficient to filter the sample using gravitational filtration, centrifugal filtration, filter stacking, sedimentation, passive filtering, or filtration using a mesh, membrane or other filtration mechanism. A filter may comprise a membrane, beads, diaphragms, colloids, weir filters, pillar filters, cross-flow filters, solvent filters, sieves, or any other filter. In some cases, the system (e.g., stool collector, collection unit, or other part of the system) may comprise geometric features that may be used to filter the stool sample or derivative thereof. The geometric features may be any useful geometry or size for filtration. The geometric features may comprise one or more geometries, e.g., pillars, pyramids, pegs, cylinders, spheres, boxes. The geometric features may comprise one or more sizes. In some cases, the geometric features comprise micropillars that may decrease in size across one or more dimensions (e.g., length, width, height, or a combination thereof) to generate a gradient of size filters. In some cases, the geometric features comprise micropillars that decrease in size across one or more dimensions to generate discrete zones of size filters. The filters may be used to filter out debris or particles larger than a specified threshold.

The collection unit may comprise one or more units to perform further processing of the stool sample. For example, the collection unit may comprise one or more apparatuses, reagents, etc., or access thereto, to perform lysis of a cell within the sample. In such cases, the collection unit may comprise any component used for cell lysis, which may be achieved using mechanical, electrical, chemical, biological, or other approaches, as described elsewhere herein. For instance, in the case of mechanical lysis, the collection unit may comprise a lysis unit (e.g., chamber or vessel) into which the stool sample or derivative thereof (e.g., homogenized stool sample) is deposited. The lysis unit may comprise a blade, an impeller, a rod, mortar and pestle, beads, or other components for mechanically lysing one or more cells in the stool sample. For example, the lysis unit may comprise a rotating impeller, which may mechanically shear or lyse the one or more cells. The lysis unit may comprise one or more beads that may be used to lyse the one or more cells. In some instances, the lysis unit comprises a combination of units for mechanical lysis; for instance, the lysis unit may comprise an impeller and a plurality of beads which are used to mechanically disrupt the sample and lyse the one or more cells in the stool sample. In other examples, the lysis unit may comprise a heating element, electrodes or other electrical components (for electrical lysis), ultrasonic wave provider, a laser, etc. In other non-limiting examples, the lysis may be performed using chemical or biological approaches, e.g., using detergents, enzymes, chaotropes, or other chemicals to lyse the one or more cells, as described elsewhere herein. A combination of lysis approaches may be used (e.g., chemical and mechanical, biological and chemical, chemical and mechanical, etc.).

The collection unit may be configured to perform an extraction, isolation, or purification of a biomolecule from the stool sample. The extraction, isolation, or purification may be performed using a variety of methods, such as affinity-based assays or separation processes (e.g., cytometry, chromatography, filtration, etc.). In some instances, beads may be used to purify, isolate, or extract the biomolecule. For instance, the bead may comprise a binding molecule such as an aptamer, biotin, avidin, streptavidin, antibodies or fragments thereof, nucleic acid molecules, etc. that can be used to capture the biomolecules (e.g., nucleic acid molecules, proteins, etc.). In some instances, the beads comprise nucleic acid molecules that are configured to capture one or more nucleic acid molecules (e.g., DNA or RNA) from the stool sample or derivative thereof (e.g., homogenized stool sample, homogenized and lysed stool sample, etc.). In such cases, the beads may be mixed with the stool sample or derivative thereof and subsequently isolated or enriched from the stool sample mixture. By way of example, the beads may be magnetic beads, and application of a magnetic force or field may be used to separate the beads (with biomolecules attached thereto) from the stool sample mixture.

In some instances, the isolation or extraction of biomolecules may be performed using multiple operations. For instance, during the isolation or extraction, multiple wash steps may be performed. Alternatively or in addition to, the isolation or extraction may include the use of a plurality of buffers, such as binding buffers, wash buffers and elution buffers, as described elsewhere herein. In such instances, the collection unit may comprise separate fluidic reservoirs or inlets and outlets to facilitate the isolation or extraction of the biomolecules.

In certain instances, the isolated biomolecule may be dispensed or otherwise separated from the stool sample (or derivative thereof). For instance, the collection unit may comprise a fluid handling system that is configured to direct the isolated biomolecule (e.g., DNA attached to a bead) into a sample container. The fluid handling system may comprise a moveable stage (e.g., a motorized stage, an automated stage, etc.). The stage may be configured to move in any direction (e.g., X-Y-Z axes). In the case when magnetic beads are used, a magnet may be used to separate or otherwise direct the isolated biomolecule into a sample container. The sample container may take any useful geometry and may comprise a tube, vial, well, plate, flask, etc. The sample container may contain an array of containers, for instance, a multi-well unit, an array configured to capture beads, etc. or an array for analyzing the biomolecule (e.g., microarray). In some instances, the collection unit comprises a multi-well unit, and each well is configured to hold a sample taken at a time point; accordingly, the multi-well unit may be amenable for performing longitudinal sample collection, in which multiple samples are collected over a period of time and distributed, at each time point, into a single well or a plurality of pre-defined wells.

In certain instances, the sample container (e.g., multi-well plate) may comprise biomolecule probes (e.g., antibodies, aptamers, nucleic acid probes, etc.). Following isolation of the biomolecules in the sample container, the probes may be used to indicate the presence, identity, quantity or other metric of the biomolecules in the stool sample. For instance, nucleic acid or protein probes may be immobilized (covalently or non-covalently) to the sample container (e.g., a surface of a well). The isolated or extracted biomolecule may bind or couple to the nucleic acid or protein probes and form a detectable product (e.g., a precipitate, a fluorescent product, a chemiluminescent or other luminescent product, etc.), optionally with the addition of other reagents (e.g., additional antibodies, fluorescent or colorimetric probes, etc.). For example, the sample container may comprise reagents or have access to reagents for performing an assay, such as fluorescence in situ hybridization (FISH) or an enzyme-linked immunosorbent assay (ELISA) or sandwich ELISA for detection of at least one biomolecule. Multiplexed assays (e.g., for measuring or analyzing different types of biomolecules such as DNA and protein) may be performed in the sample containers. In some instances, further processing may be performed in the sample containers (internally or externally of the collection unit). Non-limiting examples of processing operations include, for instance: filtration, heating, cooling, applying an electric potential, separation of one or more analytes (e.g., via filtration, chromatography, electrokinetics, centrifugation), partitioning (e.g., forming droplets), flow cytometry, merging of reagents, mixing, interfacing with an analysis instrument (e.g., mass spectrometer, sequencing instrument, etc.). The processing operations may be performed in any combination or order.

It will be appreciated that the collection unit may be configured to perform a combination of operations or processes described herein. For instance, the collection unit may be configured to receive a stool sample, homogenize the stool sample, lyse the stool sample (or lyse one or more cells in the stool sample), isolate or extract a biomolecule from the stool sample, store the isolated or extracted biomolecule (e.g., in a well of a multi-well unit) and/or analyze the isolated or extracted biomolecule. Accordingly, in some embodiments, the collection unit may act as a standalone device for processing and/or analyzing a stool sample. For instance, the collection unit may be configured to receive a stool sample from a subject, and this receipt of the stool sample may take place separately from the stool sample collection. In such cases, a stool sample may be separately obtained at a first location (e.g., at a home, a clinic, a laboratory, etc.) and analyzed or processed at a second location that can be the same or different than the first location. For example, the first location and the second location may be adjacent to or may be within a defined distance, e.g., from 1 inch apart, 1 foot apart, 1 yard apart, 1 mile apart, 1000 miles apart, etc. The first location and the second location may be at most 1000 miles apart, at most 1 mile apart, at most 1 yard apart, at most 1 foot apart, at most 1 inch apart, or less. In instances where the samples or isolated biomolecules are stored in the collection unit, analysis, characterization, or further processing may occur at yet another (third) location that may be the same or different than either the first or second locations.

The collection unit, similar to the sensing units described herein, may comprise a single housing that contains multiple compartments for performing a plurality of operations or processes described herein, or the collection unit may comprise multiple housing units that are each configured to perform one or more functions and are coupled or connected (e.g., fluidically). For example, the collection unit may comprise or be contained in a single housing, which housing includes a compartment for sample collection, a compartment for sample homogenization, a compartment for sample lysis, a compartment for isolation or extraction of biomolecules, a compartment for sample or isolated biomolecule storage, or a combination thereof. Alternatively, the collection unit may comprise multiple units, for instance, a sample collection module, a sample homogenization module, a sample lysis module, or an isolation or extraction module, which may be coupled to one another or to different modules. One or more modules may contain a separate housing. One or more modules may be coupled fluidically. For instance, the sample homogenization module may be fluidically coupled to the lysis module (e.g. via tubing, funnels, etc.), or the lysis module may be coupled to the isolation or extraction module. A module may be used to perform multiple processes; for example, the collection unit may contain a module for sample homogenization and another module to perform lysis and isolation or extraction.

The collection unit may comprise one or more sensors or sensing units. One or more sensors may include one or more electronics unit. The one or more sensors may be integrated in the collection unit, or they may be coupled to the collection unit. A sensor and/or the electronics unit may be used for detection and/or imaging of the one or more isolated or extracted biomolecules. For instance, the sensor may comprise an electro-optical sensor, which may be used to determine a spectroscopic property of the biomolecule (e.g., fluorescence, luminescence, absorbance). The sensor and/or electronics may comprise a lens, a camera, a laser, a diode (e.g., light-emitting diode), a photoconductive device, a photovoltaic, a photodiode, a phototransistor, etc. The sensor and/or electronics may be used to determine the presence or quantity of a particular biomolecule or an assortment of biomolecules. The sensor and/or electronics may comprise a micro-controller, printed circuit board, or other components. The sensor may comprise one of the processing units (e.g., microfluidic devices) described herein. The sensor may be used to automate one or more processes described herein.

As described herein, the sensor or the electronics unit of the sensor may comprise a communication interface that allows for transmitting and/or receiving data corresponding to the parameter of the stool sample or isolated or extracted biomolecule. In some cases, the data may be transmitted to an electronic device (e.g., via the electronics unit) in communication with the communication interface. The communication interface may be a wireless communication interface, e.g., a Wi-Fi interface, a near-field communication interface, or a Bluetooth interface. The electronic device may be a device that may communicate with the communication interface. The electronic device may be a mobile device (e.g., a smart phone, tablet, laptop, etc.). Alternatively or in addition to, the communication interface may be a wired communication interface. The sensor may comprise a port for communication and/or a power supply (e.g., universal serial bus (USB), USB-type C, Thunderbolt, Ethernet, etc.).

The collection unit may be configured to clean one or more components within the collection unit. In such cases, the collection unit may comprise a cleaning or sterilization component. One or more reagents may be used for sterilization of a part of the system (e.g., the collection unit or sub-compartments therein.). In such cases, the sterilization reagents may comprise any suitable sterilization reagent, e.g., bleach, chlorine, antimicrobial agents, alcohol, an acid, a base, minerals, soap, etc. or a combination thereof. Sterilization of the collection unit may be performed automatically or manually.

FIG. 9 provides an example rendering of a system comprising a stool collector and collection unit as described herein. The stool collector 905 may be coupled to a toilet or may be a part of a toilet. In some instances, the stool collector 905 may toggle between an “open” and “closed” configuration, in which the “open” configuration enables capture or collection of a sample (e.g., via extension of a catchment unit toward the toilet bowl). The configurations may be toggled based on an input from a sensor (not shown) that may be integrated with the stool collector 905 and may detect the presence of a subject or a sample ready for collection (e.g. via an optical or weight sensor). The stool collector 905 may be coupled (e.g., via a fluid flow path) to a collection unit 910. In some instances, the stool collector 905 may be used to collect the stool sample from the subject and homogenize the collected stool sample; in other instances, the homogenization may occur within the collection unit 910. The collection unit 910 may be removably coupled to the stool collector 905, or the stool collector 905 and the collection unit 910 may be integrated as a unit.

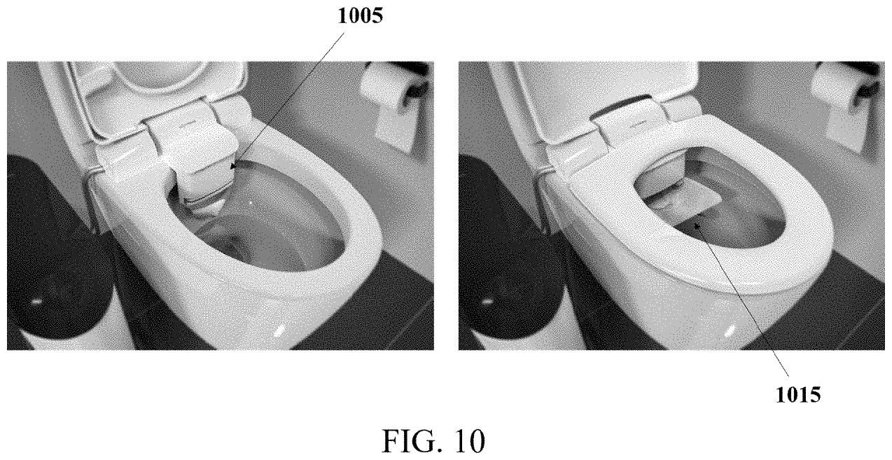

FIG. 10 provides an example rendering of a stool collector, such as the stool collector shown in FIG. 9. The stool collector 1005 may be configured to collect the stool sample from the subject. As described herein, the stool collector 905 may toggle between an “open” (e.g., right panel) configuration and a “closed” (e.g., left panel) configuration, in which the “open” configuration enables capture or collection of a sample (e.g., via extension of a catchment unit toward the toilet bowl). The catchment unit 1015 is depicted as a tray in FIG. 10, but the catchment unit 1015 may take any useful geometry, e.g., arms, rods, scoops, fingers, nets, sieves, etc. In certain instances, the catchment unit 1015 comprises a membrane that may be deformable during extension or retraction.

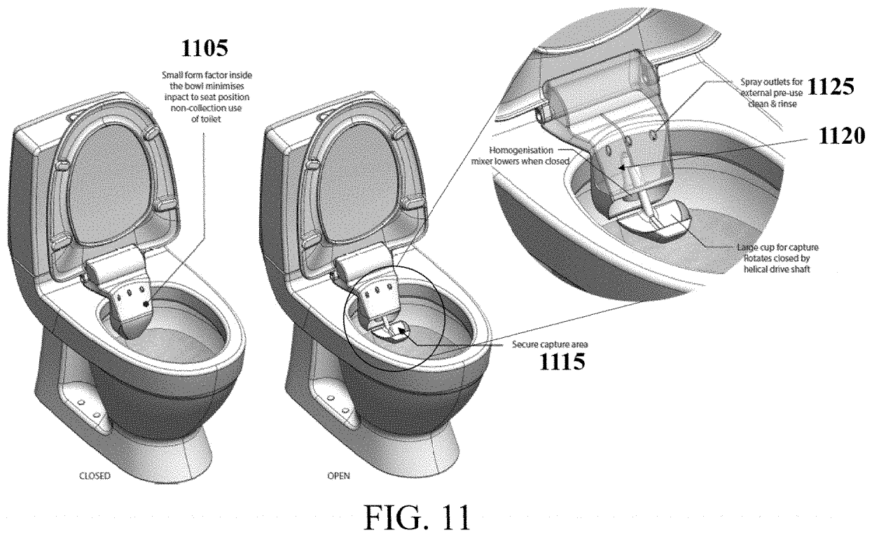

FIG. 11 provides a schematic of another example of a stool collector, such as the stool collector shown in FIG. 9. The stool collector 1105 may be configured to collect the stool sample from the subject. As described herein, the stool collector 1105 may toggle between an “open” (e.g., middle panel) configuration and a “closed” (e.g., left panel) configuration, in which the “open” configuration enables capture or collection of a sample (e.g., via extension of a catchment unit toward the toilet bowl). The catchment unit 1115 may comprise a cup or lip area that facilitates capture of the stool sample. The catchment unit 1115 may extend toward the subject or the toilet bowl via an extension mechanism (e.g., a helical drive shaft) during the transition to the “open” configuration. The stool collector 1105 may additionally comprise a chamber 1120, which may be used for homogenization of the stool sample (e.g., when the stool collector 1105 is in a “closed configuration”). The chamber 1120 may comprise appendages or apparatuses for homogenization, e.g., blades, mixers, rods, fluid inlets, etc. The stool collector 1105 may additionally comprise outlets 1125, which may be used to direct a fluid (e.g., water, a liquid, or a solution) to a portion of the stool collector 1105 or toward the toilet bowl, which may be used to homogenize the sample (if directed toward the chamber 1120), to clean or sterilize the stool collector 1105 or the toilet bowl, or for other purposes (e.g., dilution, mixing, or spraying).

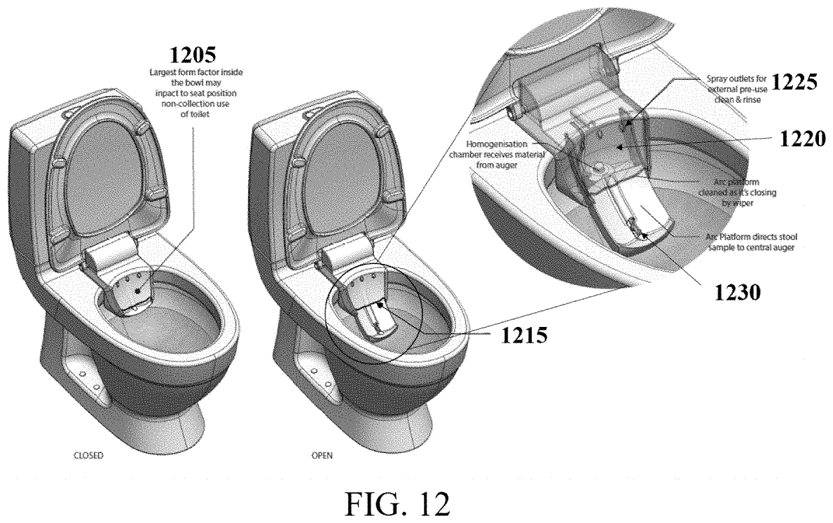

FIG. 12 provides a schematic of another example of a stool collector, such as the stool collector shown in FIG. 9. The stool collector 1205 may be configured to collect the stool sample from the subject. As described herein, the stool collector 1205 may toggle between an “open” (e.g., middle panel) configuration and a “closed” (e.g., left panel) configuration, in which the “open” configuration enables capture or collection of a sample (e.g., via extension of a catchment unit toward the toilet bowl). The catchment unit 1215 may comprise a platform, which may be arc-shaped, substantially planar, or any other geometry. The catchment unit is configured to capture or collect the stool sample. The catchment unit 1215 may extend (or fold out) toward the subject or the toilet bowl via an extension mechanism (e.g., an auger 1230) during the transition to the “open” configuration. The stool collector 1205 may additionally comprise a chamber 1220, which may be used for homogenization of the stool sample (e.g., when the stool collector 1205 is in a “closed configuration”). The chamber 1220 may comprise appendages or apparatuses for homogenization, e.g., blades, mixers, rods, fluid inlets, etc. In some instances, the auger 1230 is used to transport the stool sample to the homogenization chamber 1220. The stool collector 1205 may additionally comprise outlets 1225, which may be used to direct a fluid (e.g., water, a liquid, or a solution) to a portion of the stool collector 1205 or toward the toilet bowl, which may be used to homogenize the sample (if directed toward the chamber 1220), to clean or sterilize the stool collector 1205 or the toilet bowl, or for other purposes (e.g., dilution, mixing, or spraying).

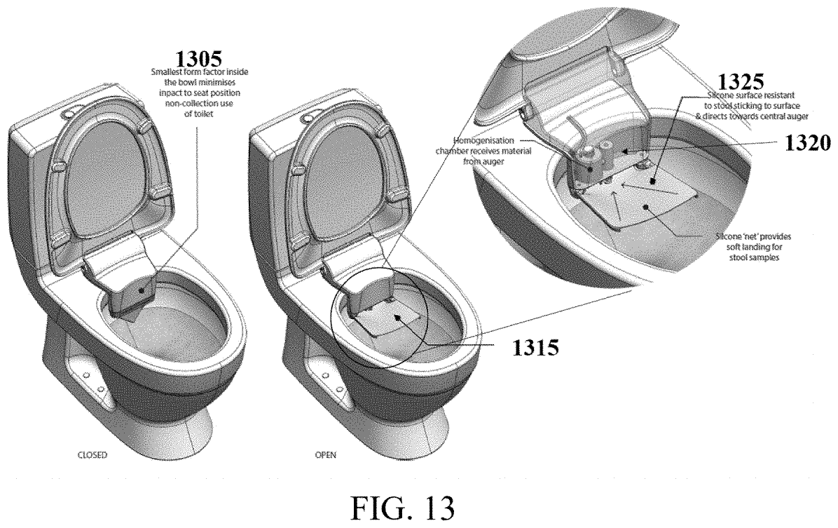

FIG. 13 provides a schematic of another example of a stool collector, such as the stool collector shown in FIG. 9. The stool collector 1305 may be configured to collect the stool sample from the subject. As described herein, the stool collector 1305 may toggle between an “open” (e.g., middle panel) configuration and a “closed” (e.g., left panel) configuration, in which the “open” configuration enables capture or collection of a sample (e.g., via extension of a catchment unit toward the toilet bowl). The catchment unit 1315 may comprise a substantially planar platform. In some instances, the planar platform comprises a membrane or compliant substrate (e.g., silicone). The catchment unit 1315 may extend toward the subject or the toilet bowl via an extension mechanism during the transition to the “open” configuration. The stool collector 1305 may additionally comprise a chamber 1320, which may be used for homogenization of the stool sample (e.g., when the stool collector 1305 is in a “closed configuration”). The chamber 1320 may comprise appendages or apparatuses for homogenization, e.g., blades, mixers, rods, fluid inlets, etc. The stool collector 1305 may additionally comprise outlets 1325, which may be used to direct a fluid (e.g., water, a liquid, or a solution) to a portion of the stool collector 1305 or toward the toilet bowl, which may be used to homogenize the sample (if directed toward the chamber 1320), to clean or sterilize the stool collector 1105 or the toilet bowl, or for other purposes (e.g., dilution, mixing, or spraying).

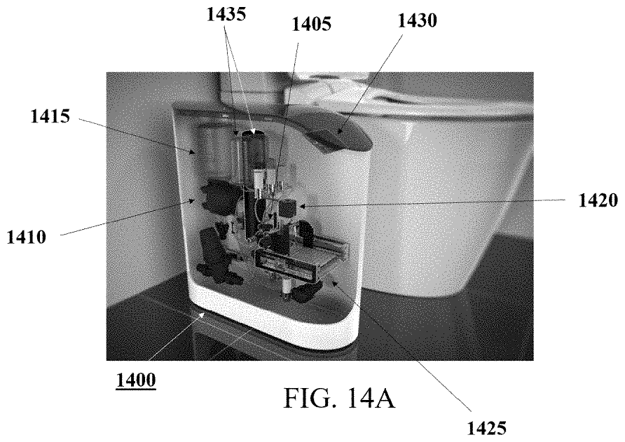

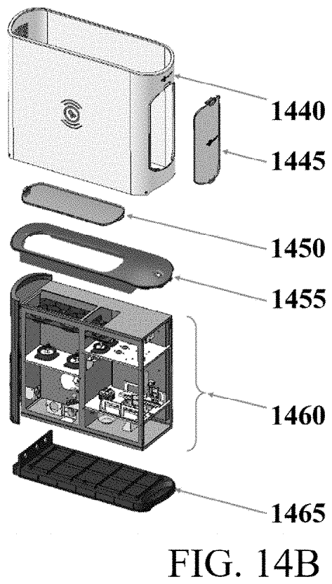

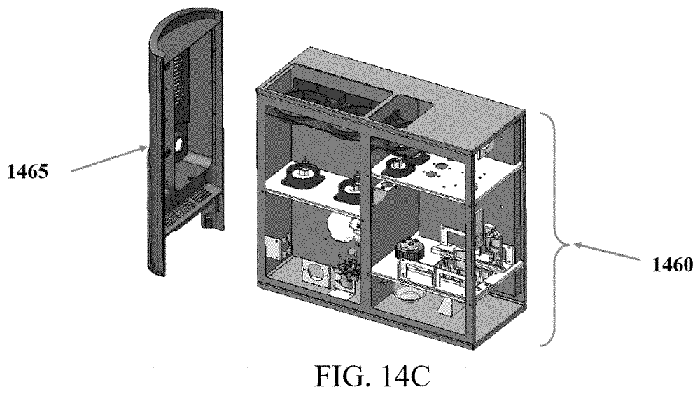

FIG. 14 schematically illustrates a collection unit, as described herein. FIG. 14A shows an integrated system. The collection unit 1400 may be coupled to the stool collector (not shown) or may be separate from the stool collector. The collection unit may comprise multiple subsystems or modules, and in some instances, may be alterable or modular. For instance, the collection unit 1400 may comprise a unit that is configured to perform lysis of a cell within the stool sample. The lysis unit may comprise, for instance, a chamber 1405 into which the stool sample (or derivative thereof) is input. The chamber 1405 may configured to also contain one or more lysis beads (not shown), which may be used to lyse the cell (or cells) within the stool sample (or derivative thereof). In some instances, the chamber 1405 comprises an impeller that is used to rotate and disperse the beads, as well as aid in mechanical lysis of the cell (or cells). The chamber 1405 may also comprise a variety of fastening mechanisms, chambers, filters, motors, gaskets, valves, spacers, fittings, or other parts to facilitate fastening of the parts to one another and to facilitate cell lysis.

The collection unit 1400 may comprise one or more units to perform an extraction, isolation, or purification of a biomolecule from the stool sample (or derivative thereof). In one particular example, nucleic acid molecules (e.g., DNA or RNA) may be isolated or extracted using beads with capture nucleic acid molecules, e.g., silica magnetic beads with capture sequences (not shown). The stool sample or derivative thereof (e.g., the lysed stool sample) may be directed to a processing chamber 1410, which may comprise or be configured to contain the silica magnetic beads. The processing chamber 1410 may be coupled to one or more fluid reservoirs 1415, which may be used for performing a reaction, and which may include, for instance, binding buffers, wash buffers, elution buffers, etc. The beads may be used to isolate the nucleic acid molecules from the stool sample or derivative thereof.

In some instances, the collection unit 1400 comprises a dispensing and storage unit. For instance, the dispensing unit 1420 may comprise a fluid handling system that may be used to direct a fluid or a sample in a controlled manner to a location of interest. By way of example, the dispensing unit 1420 may be used to direct the sample or derivative thereof (e.g., isolated biomolecules, which may be attached to beads) to a sample container 1425. The sample container 1425 may comprise an array of containers, such as a multi-well unit, a plurality of vials, tubes, or other containers. In some instances, a force may be applied to the isolated biomolecules (which, in some instances, are attached to beads) to direct the isolated biomolecules to the sample container or a portion thereof. Alternatively or in addition to, the isolation (or additional isolation or extraction processes) may be performed in the sample container 1425. For example, a gravitational force, hydraulic force, centrifugal force, inertial force, or positive or negative pressure may be applied to direct the beads to one or more wells of a multi-well unit. Alternatively or in addition to, the isolated biomolecule may be eluted or removed from the bead (e.g., in processing chamber 1410), and the eluted, isolated biomolecule may be directed into the sample container 1425. In cases where a magnetic bead is used to isolate the biomolecule, a magnet (not shown) may be coupled to or placed adjacent to the sample container (e.g., multi-well unit). The collection unit may also comprise container sealers (e.g., plastic sealer films, gaskets, lids, etc.) to seal the isolated biomolecule in the sample container or portion thereof (e.g., individual well of a multi-well unit).

The collection unit 1400 may comprise a sensor and/or one or more electronics. For instance, the collection unit may comprise a printed circuit board (PCB) 1430 or micro-controller. The PCB may comprise a master PCB in communication with a slave PCB. In some instances, the master PCB may be in communication with a slave PCB of the stool collector (not shown). The PCB may be used to receive one or more inputs and to output one or more signals, e.g., for relaying instructions to one or more components for executing an action (e.g., for fluid handling of the sample or derivatives thereof, for automatic cleaning, etc.).

The collection unit 1400 may be configured to automatically clean one or more components of the collection unit. For instance, the collection unit may comprise reservoirs 1435 containing water, a solution, a buffer, or a sterilization or cleaning agent (e.g., alcohol, bleach, acids, bases, etc.). One or more reservoirs 1435 may be used to accommodate any number of useful solutions; for example, one reservoir may contain filtered water and the other may contain bleach. In some instances, another reservoir or container may be used for mixing reagents (e.g., to generate a diluted bleach solution), which may be used for sterilization or cleaning of one or more fluid flow paths of the collection unit. For example, the fluid handling system 1420 may be sterilized after each deposit in the sample container 1425. Similarly, the processing chamber 1410, or any fluidic connections thereto/therefrom may be sterilized. Sterilization of the fluid flow paths or connections may be useful in preventing cross-contamination (e.g., in the instance that multiple subjects use the same collection unit or across varying time points of sample collection).

In some instances, although not shown in FIG. 14, the collection unit may comprise a homogenization chamber (not shown), in which a stool sample may be deposited. The homogenization chamber may be coupled to one or more fluid inlets and/or outlets, and any apparatuses used for homogenizing the stool sample, as described elsewhere herein. For instance, the collection unit may comprise blades, water pressurizers, beads, mortars, and any necessary fluid inlets and outlets and other mechanical items (e.g., valves, pumps, etc.) for homogenization of the sample.

It will be appreciated that the collection unit may comprise any combination of components and units. For instance, the collection unit may comprise a homogenization chamber and a sample processing unit, or the collection unit may comprise a sample processing unit and a fluid handling system for depositing the stool sample (or derivative thereof) into a sample container. In some instances, the collection unit comprises a sample processing unit (e.g., a unit for performing cell lysis), a unit for isolating one or more biomarkers from the stool sample or derivative thereof, a storage or sample container unit, and a cleaning or sterilization unit for cleaning one or more components of the collection unit. Any one or combination of these processes may be automated and performed without a user intervention. In some instances, certain processes are performed automatically and others are performed manually. In some instances, certain processes may be manually or automatically performed. In some instances, one or more operations (e.g., sample processing, lysis, isolation or extraction of a biomolecule, storage, analysis, cleaning of components, etc.) may be repeated one or more times without a user intervention. For instance, multiple stool samples may be collected and processed sequentially, such that no user or human involvement is required. In some instances, any of the processes described herein may be repeatable, without user interaction, for at least 1 cycle, at least 5 cycles, at least 10 cycles, at least 20 cycles, at least 30 cycles, at least 40 cycles, at least 50 cycles, at least 60 cycles, at least 70 cycles, at least 80 cycles, at least 90 cycles, at least 100 cycles, at least 200 cycles, at least 300 cycles, at least 400 cycles, at least 500 cycles, at least 600 cycles, at least 700 cycles, at least 800 cycles, at least 900 cycles, at least 1000 cycles, or more. In such instances, the collection unit may comprise reservoirs comprising reagents for sample processing that do not require refilling for the designated number of cycles. Alternatively or in addition to, the reagents may be provided in replaceable cartridges. In such instances, the sensing unit or sensor of the collection unit may comprise a sensor that detects the volume of reagents in the replaceable cartridges and may alert (e.g., via a communication interface or other alert system (e.g., audio or visual output)) the subject or other user when the replaceable cartridges have been depleted and require replacement.

As described herein, the collection unit may comprise one or more housings. FIG. 14B and FIG. 14C schematically show an example housing unit of the collection unit. The housing may comprise, for instance, a main casing or shroud 1440, and one or more doors (e.g., 1445 and 1450) which may allow access to the individual compartments (e.g., lysis unit, storage unit, reagents, reservoirs, reagent cartridges, etc.). The housing may additionally comprise one or more covers (e.g., 1455 and 1465). A main chassis 1460 may house structurally support one or more components of the collection unit. The main chassis 1460, as shown in FIG. 14C, may be coupled to a removable cover 1465, which may allow additional access to the compartments within the housing.

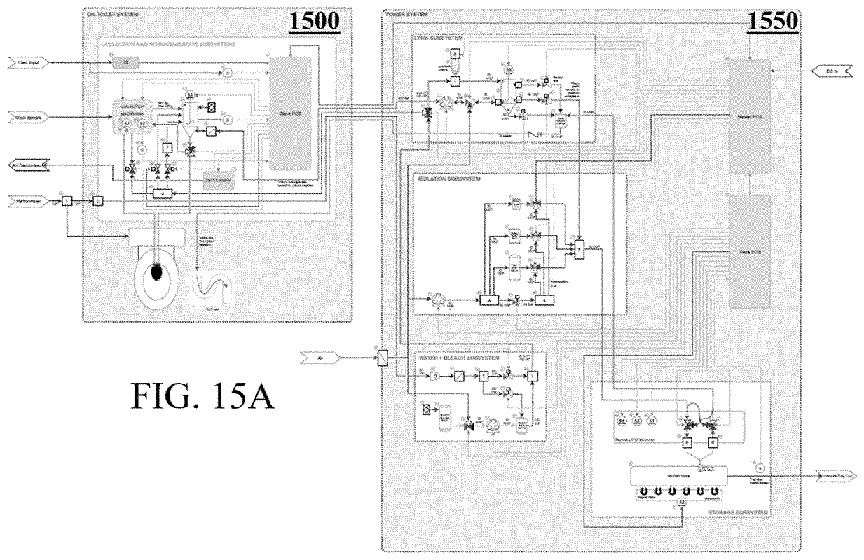

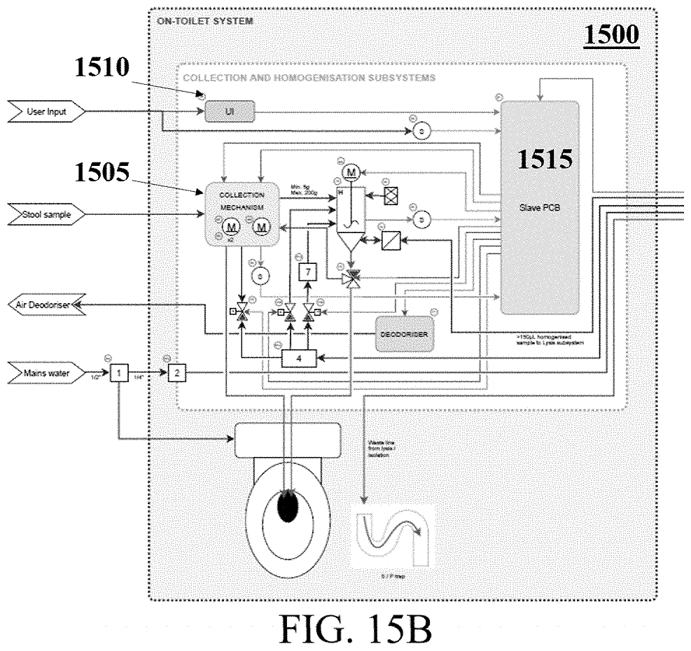

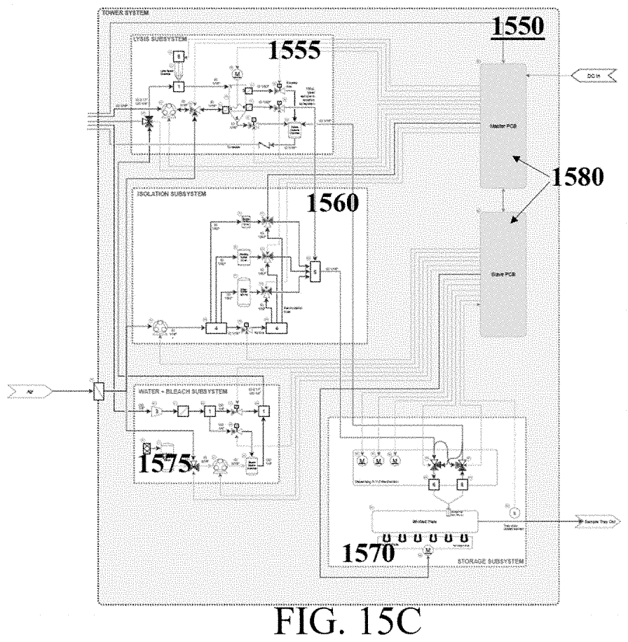

FIG. 15 provide diagrams of an example system architecture comprising a stool collector, which may be coupled to or configured to couple to a toilet, and a collection unit. FIG. 15A shows an example system architecture of the stool collector and collection unit. FIG. 15B shows an enlarged view of the example architecture of the stool collector shown in FIG. 15A. FIG. 15C shows an enlarged view of the example architecture of the collection unit shown in FIG. 15A.

Referring to FIGS. 15A-B, the stool collector 1500 may be coupled (e.g., fluidically, electrically) to the collection unit 1550. The stool collector 1500 may comprise a collection mechanism 1505 (e.g., catchment unit, as described herein) for collecting the stool sample from a subject. In some instances, the stool collector 1500 comprises a user interface 1510 that is configured to receive input of or from a user (e.g. from a sensor about the proximity of the subject to the stool collector, a manual input for activation, etc.). The user interface 1510 may be coupled to a PCB 1515. The stool collector 1500 may also comprise a plurality of inlets and outlets, e.g., to provide the stool sample, water or other reagents, deodorizers, etc. In some instances, the stool collector 1500 comprises a mixer or agitator or other mechanisms for homogenization of the sample (e.g., beads). The homogenized stool sample may then be directed to the collection unit 1550.

Referring to FIG. 15C, the collection unit 1550 may comprise one or more units to process the stool sample (or derivative thereof) and/or to isolate the one or more biomolecules. For instance, the collection unit 1550 may comprise a lysis unit 1555, an isolation unit 1560 (for isolation or extraction of one or more biomolecules), a storage unit 1570, and a cleaning or sterilization unit 1575. The collection unit 1550 may also comprise one or more PCBs 1580. The lysis unit 1555 may comprise a chamber, in which the stool sample or derivative thereof (e.g., homogenized stool sample) may be added. The chamber may also comprise mechanisms for lysis (e.g., of a cell in the stool sample or derivative thereof), e.g., an impeller and beads, which can be made of ceramic, metal, glass, plastic, or another material, and any fluid connections to facilitate lysis. Waste products may be generated and directed to a waste chamber.

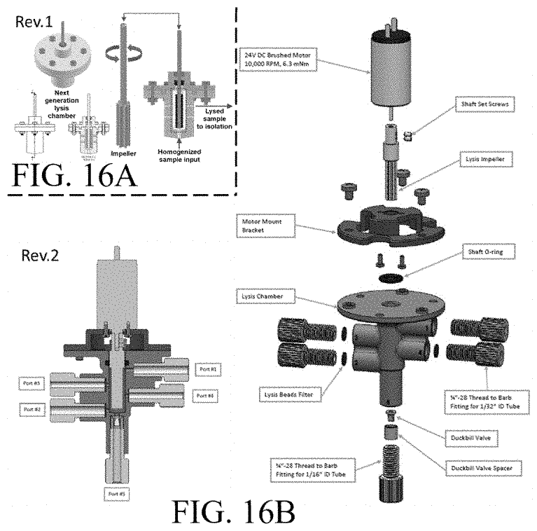

By way of example, the stool sample may be subjected to conditions sufficient to lyse one or more cells in the stool sample or derivative thereof. FIG. 16 schematically shows two examples of a lysis system, which may be included in the stool collector or the collection unit. FIG. 16A shows a lysis system that comprises a lysis chamber and a rotatable impeller. The impeller may be used to rotate, mix, or otherwise move the stool sample (e.g., homogenized stool sample). Optionally, beads may be used to further process the sample and lyse the cells contained therein. In some instances, other lysis approaches may be used in conjunction or alternatively to the system presented in FIG. 16A. For instance, chemical reagents, such as detergents or surfactants may be used to aid in achieving cell lysis.

FIG. 16B illustrates schematically another example lysis system that also comprises a lysis chamber and a rotatable impeller. The lysis system may comprise a plurality of parts, including but not limited to fastening parts (e.g., screws, gaskets, o-rings), valves, filters, spacers, etc. The impeller may be rotated manually within the lysis chamber or using a motor (e.g., a 24V DC motor) at any suitable angular velocity. For example, the motor may be configured to rotate the impeller at approximately 100, 1000, 10000, 100000 or more revolutions per minute (RPM). The chamber may comprise a plurality of ports for introduction of reagents (e.g. buffers, lysis reagents, water, solutions, etc.). In some instances, beads may be used to facilitate lysis of the one or more cells in the stool sample or derivative thereof. In such cases, the beads may be introduced into a lysis chamber, optionally with a rotatable impeller for moving the beads.

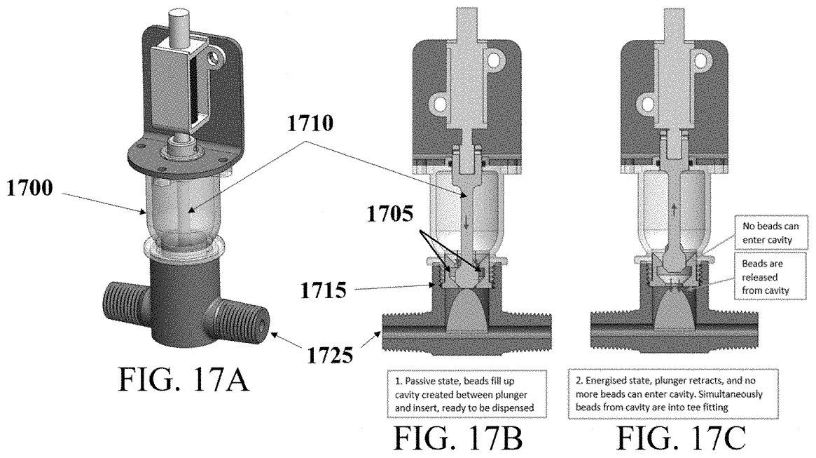

For example, FIG. 17 illustrates an example mechanism for dispensing of beads into the lysis unit (e.g., as shown in FIG. 16). FIG. 17A shows a perspective view of the bead dispensing unit, and FIGS. 17B-C show cross-sectional views of the bead dispensing unit. The bead dispensing unit may comprise a chamber 1700 comprising one or more beads, which can be made of one or more materials, e.g., ceramic (e.g., silicon, zirconium), metal, glass, plastic, etc. and a plunger 1710. FIG. 17B shows the bead dispensing unit in an “open configuration.” In such a configuration, the plunger 1710 may be disposed at a first position relative to (e.g., substantially flush with) a fitting or insert 1715. The fitting or insert 1715 may comprise one or more cavities 1705 into which beads may enter when the plunger 1710 is flush (in the “open configuration”). FIG. 17C shows the bead dispensing unit in a “closed configuration.” In such a configuration, the plunger 1710 may be disposed at a second position relative to the fitting or insert 1715. In such a second position, the plunger 1710 may substantially obstruct beads from entering the one or more cavities 1705. The beads may then enter another fitting 1725, which may be connected to the lysis chamber (e.g., as shown in FIG. 16).

In some cases, as described herein, lysis of a cell within the stool sample or derivative thereof may be achieved chemically or biologically via addition of a chemical (e.g., detergent, chaotropes) or a biological agent (e.g., enzyme). For example, a detergent that is ionic (e.g., sodium or sarcosyl dodecyl sulfate), and/or a non-ionic detergent (e.g., Triton-X 100, Tween-20, CHAPS) may be used to lyse the cells. Reducing agents or other denaturants, e.g., 2-mercaptoethanol, dithiothreitol, TCEP, TCEP-HCl, or variants thereof may also be used in lysis. Similarly, the components of any buffers (e.g., binding buffers) for the beads may be used, e.g., guanidine thiocyanate, EDTA, Tris, alcohols, sodium acetates, or acids or bases (for adjusting buffer pH).

Following lysis, and referring again to FIGS. 15 A-C, the lysed stool sample may be directed to the isolation unit 1560. The isolation unit 1560 may comprise one or more chambers for isolating or extracting a biomolecule from the lysed stool sample. For instance, the biomolecule may be extracted using one or more beads (e.g., beads with nucleic acid molecules, antibodies, or other binding moieties that can capture the biomolecule). In such an example, the isolation subsystem may comprise one or more reservoirs, which may contain buffers for performing one or more reactions, e.g., for binding the biomolecule to the bead, washing the beads, eluting the biomolecule from the beads, etc. Accordingly, the one or more reservoirs may comprise a binding buffer, a wash buffer, or an elution buffer. In some instances, the biomolecule may be extracted or isolated in the storage unit 1570. In such cases, the storage unit may have fluidic access to the reservoirs containing the binding buffer, wash buffer, and/or elution buffer.

The processed stool sample, the beads comprising the isolated or extracted biomolecules, or the isolated or extracted biomolecules alone (e.g., eluted from a bead), may be directed to a storage unit 1570. The storage unit may comprise one or more sample containers, e.g., a multi-well unit (e.g., a 6-well, 12-well, 24-well, 36-well, 48-well, 96-well, 384-well, etc. plate), or a plurality of cartridges. In instances when the beads are magnetic, a magnet may be applied to a surface or region of the plate, which may help direct the bead to the sample container (e.g., a well of a multi-well unit). In other instances, another force (e.g., gravitational, hydrodynamic, centrifugal, etc.) may be used to direct the bead or the extracted biomolecules to the sample container. For instance, a fluid handling unit, optionally with a moveable (e.g., motorized) stage, may be used to dispense the beads or isolated biomolecules into the one or more sample containers. In some instances, the isolation of the biomolecule is performed in the storage unit 1570.

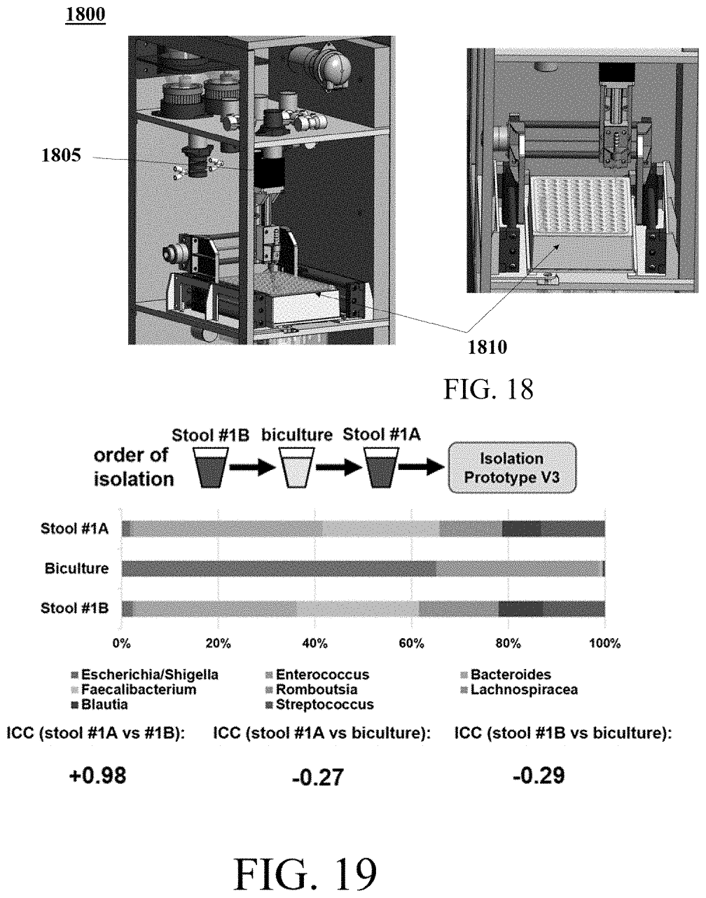

FIG. 18 shows an example storage unit. The storage unit 1800 may comprise a handling unit 1805, which may comprise a moveable stage (e.g., in the X, Y, and Z directions). The stage may use a motor and may be in fluid communication with the isolation or extraction unit. Following isolation or extraction of the biomolecules (e.g., using a bead based approach), the isolated or extracted biomolecules may be directed to the handling unit 1805, which can be used to deposit or dispense the sample (or isolated or extracted biomolecules) in a sample container 1810. In some instances, the handling unit 1805 comprises a fluid handling system, which may fluidically dispense the sample (or isolated or extracted biomolecules) in the sample container 1810. In other instances, a force (e.g., gravitational, magnetic, optoelectronic, centrifugal, etc.) may be used or applied to dispense the sample to the sample container 1810. For instance, the isolated or extracted biomolecules may be attached or coupled to magnetic beads, and a magnet may be placed adjacent to the sample container 1810, which may direct the beads into the sample container 1810. The sample container 1810 may comprise multiple partitions (e.g., well, flasks, vials, tubes, etc.). In such instances, the sample (or isolated or extracted biomolecules) may be placed in an individual partition. The individual partitions may be individually addressable, e.g., by the subject or a user. For instance, the individual partitions may be programmed (e.g., using an electronic device in communication with the stool collector or collection unit), such that each sample collected is distributed in a pre-determined partition.

In some instances, one or more components or the entire collection unit is stored at ambient (room) temperature. In such instances, the one or more extracted biomolecules, as well as any other reagents stored in the collection unit (e.g., in a reservoir) may be stored at ambient temperature for a prolonged period (e.g., several days, weeks, months, or years). In some instances, further processing of the isolated or extracted biomolecules may be performed. For instance, the isolated or extracted biomolecules may be desiccated or subjected to drying, or may be stored with preservatives, such as those described herein.

For instance, it may be useful to store the extracted or isolated biomolecule prior to, during, or following sample processing and/or analysis. In some cases, the extracted or isolated biomolecule may be stored for further analysis. In some cases, the storage of extracted or isolated biomolecule may occur following analysis of the extracted or isolated biomolecule and the extracted or isolated biomolecule may be subsequently re-analyzed. The extracted or isolated biomolecule may be configured to be activated or reconstituted following storage, e.g., via heating or cooling, resuspension in a solution or buffer, etc. The storage of the extracted or isolated biomolecule may comprise drying, chilling, freezing, fermenting, curing, lyophilizing, heating, pasteurizing, or adding preservatives or stabilization reagents to the extracted or isolated biomolecule (e.g., in the storage unit). Non-limiting examples of preservatives or stabilization reagents include: formaldehyde, paraformaldehyde, tert-butylhydroquinone, antimicrobial agents, e.g., sorbic acid, sodium sorbate, benzoic acid, sodium benzoate, hydroxybenzoate, sulfur dioxide, sulfites, nitrite, nitrate, lactic acid, propionic acid, isothiazolinones, phenol derivatives, ascorbic acid, butylated hydroxytoluene, gallic acid, tocopherols, etc. In some instances, the extracted or isolated biomolecule, or a bead containing a biomolecule bound thereto, may be stored in one or more individual cartridges, which may be individually addressable and/or removed from the collection unit.

In some instances, the sample container (e.g., wells, vials, cartridges, multi-unit arrays, etc.) may be collected (i.e., may be removable) from the collection unit. In such instances, further processing of the samples (or extracted biomolecules) may be performed, either inside the sample container or outside the sample container. For instance, the further processing may comprise characterization, analysis, or identification of the sample and can include processes such as imaging or microscopy, a biochemical assay (e.g., immunoassay, a nucleic acid assay (e.g., fluorescence in situ hybridization), sequencing, PCR, or another nucleic acid analysis technique, as described herein. The analysis or characterization may comprise protein or peptide analysis, e.g., mass spectrometry, nuclear magnetic resonance, Raman spectroscopy, etc. as described elsewhere herein. A combination of techniques may be used. For example, the sample or isolated biomolecule may be imaged and subjected to a biochemical assay (e.g., sequencing, PCR, immunoassays, etc.).

The collection unit may comprise a cleaning or sterilization unit (e.g., 1575 of FIG. 15C). The sterilization unit may comprise one or more containers and/or reservoirs (e.g. containing a cleaning or sterilization reagent, e.g., hydrogen peroxide, bleach, alcohol (e.g., ethanol, isopropanol), etc. In some instances, the cleaning or sterilization unit comprises a chamber for diluting or mixing reagents (e.g., for preparation of a diluted bleach solution). The sterilization agents may be used anywhere in the collection unit and may be useful in preventing cross-contamination across samples. For instance, the sterilization agents may be used in the lysis unit, the isolation or extraction unit, or the storage unit. The sterilization agents may be flushed through the entire collection unit, or a portion thereof (e.g., particular fluid lines connecting the units).

In some instances, the beads for lysis and/or extraction or isolation may be reusable. The beads may be automatically replaced in the collection unit (e.g., using a bead chamber with plunger mechanism), or the beads may be sterilized and reused. For instance, the beads, following lysis or extraction (or isolation), may be returned to a different compartment of the collection unit, or a portion of the stool collector (e.g., homogenization chamber). The beads may then be subjected to sterilization (e.g., using heat, UV, sterilization agents such as water and bleach) and can be re-used.

The collection unit (e.g., 1550) and the components therein may comprise any part that is useful in the processes described herein. For instance, the collection unit may comprise one or more, or a combination of: a splitter, an adapter, a pressure adjuster (e.g. focuser or reducer), a manifold, a dispensing unit or dispensing needle, a flow restrictor, a solenoid, a valve (e.g., manual valve, 2-way valve, 3-way valve, etc.), a filter (e.g., air or liquid filter), a motor, a pump, a spacer, a gasket, a storage vessel or container, a metering object, a mixer or agitator, a sensor, a magnet, fluid lines, electric lines, etc. One or more units may be provided in a microfluidic format. One or more units may be connected fluidically and/or electrically. In some instances, the collection unit comprises one or more electronics (e.g., PCBs) which may be used to execute or relay commands or instructions to a component of the collection unit or stool collector or to automate a process.

### Systems for Stool Processing and Analysis

Also disclosed herein are systems for processing or analyzing a stool sample of a subject. The system may comprise a stool collector configured to couple to a toilet and a sensing unit comprising a sensor comprising an electronics unit, wherein the sensing unit is coupled to or configured to couple to the stool collector. In some instances, the stool collector is configured to collect the stool sample of the subject. In some instances, the sensing unit is configured to receive the stool sample or derivative thereof collected from the subject. The electronics unit may be configured to process the stool sample or derivative thereof, and the sensor may be configured to analyze the stool sample or derivative thereof, or a chemical or biological material within the stool sample.

In another aspect, disclosed herein is a system for processing a stool sample of a subject, comprising: a stool collector configured to couple to a toilet and a collection unit coupled to or configured to couple to the stool collector. The stool collector may be configured to collect the stool sample of the subject, and the collection unit may be configured to extract or isolate a biomolecule from the stool sample or derivative thereof.

As described herein, the stool collector may be coupled to or configured to couple to the sensing unit through a fluid flow path. The stool collector may be coupled to a number of parts, such as the toilet or the collection unit. The stool collector may be removably coupled to the toilet, non-removably coupled to the toilet, or be a part of the toilet. The stool collector may be removably coupled to the collection unit, non-removably coupled to the collection unit, or be a part of the collection unit. In certain instances, the stool collector is detachable or removable from the toilet and/or the collection unit. For instance, the stool collector may be coupled to the toilet via one or more fastening mechanisms (e.g., via an interference fit, force fit, shrink fit, location fit, etc. Other fastening mechanisms are possible, such as form-fitting pairs, hooks and loops, latches, threads, screws, staples, clips, clamps, prongs, rings, brads, rubber bands, rivets, grommets, pins, ties, snaps, Velcro, vacuum, seals, or a combination thereof. Alternatively or in addition to, the stool collector may be coupled to the collection unit via one or more fastening mechanisms.

In some instances, the collection unit and/or the sensing unit may be connected to the stool collector and/or toilet via an external part. The collection unit may comprise one or more sensors or a sensing unit. The collection unit may be connected to the stool collector via a fluid flow path (see, e.g., FIG. 9). The part or fluid flow path may comprise any useful connector such as tubing, vessels (e.g., tubes, containers, vials, flasks), valves, clamps, clips, funnels, etc. In some cases, the stool sample or derivative thereof (e.g., homogenized sample) may be directed to the collection unit via tubing and one or more pumps. For example, following the collection of the stool sample or derivative thereof (e.g., homogenized stool sample), the stool sample or derivative thereof may be directed to the collection unit, which may be coupled to the stool collector via tubing and a pump. In such cases, the stool sample or derivative thereof may be pumped through the tubing to the collection unit. The stool sample may be directed to the collection unit via one or more forces, e.g., positive pressure, negative pressure (e.g., aspiration), centrifugal, capillary, gravitational, frictional, electric, magnetic forces. In some cases, the stool sample or derivative thereof may be directed to the collection unit without intermediary vessels. In such cases, the stool sample or derivative thereof may be directed to the collection unit via one or more forces, e.g., positive pressure, negative pressure (e.g., aspiration), centrifugal, capillary, gravitational, frictional, electric, magnetic forces.

In some instances, the collection unit is separate from the stool collector or removably coupled to the stool collector. For instance, the collection unit may be connected to the stool via removable tubing or pipes, such that following collection, the stool sample is deposited or otherwise directed (e.g., via a fluid flow path) to the collection unit, where further processing may be performed, either at the site of collection or elsewhere (e.g., at a clinic or laboratory). In other instances, the collection unit may be provided alone, separate from the stool collector. In such instances, a stool sample (which may be provided separately) may be deposited or directed to the collection unit, which can be used for further processing.

As described herein, the sensor or the electronics unit of the sensor or sensing unit may comprise a communication interface that allows for transmitting and/or receiving data corresponding to the parameter of the stool sample or isolated or extracted biomolecule. In some cases, the data may be transmitted to an electronic device (e.g., via the electronics unit) in communication with the communication interface. The communication interface may be a wireless communication interface, e.g., a Wi-Fi interface, a near-field communication interface, or a Bluetooth interface. The electronic device may be a device that may communicate with the communication interface. The electronic device may be a mobile device (e.g., a smart phone, tablet, laptop, etc.). Alternatively or in addition to, the communication interface may be a wired communication interface. The sensor may comprise a port for communication and/or a power supply (e.g., universal serial bus (USB), USB-type C, Thunderbolt, Ethernet, etc.).

The electronics unit of the sensor may comprise one or more electronics. The electronics unit may comprise a microcontroller, a detection system, a sensor, a communication interface, or a combination thereof. An electronics unit may be integrated with a plurality of sensors or may be coupled to the one or more sensors. The electronics may be used for information processing, signal processing, and may comprise any suitable part including, but not limited to: vacuum tubes, transistors, diodes, integrated circuits, electrical components, optoelectronics, wires, motors, generators, batteries, switches, relays, transformers, resistors, transmitters, receivers. The electronics may comprise analog circuits, digital circuits, or a combination thereof. The electronics unit may comprise units that are configured to integrate with other analysis units, e.g., sequencing units, mass spectrometer, FTIR, etc. The electronics unit may comprise electrodes, electrical inlets, or electrical units that comprise or are configured to couple to electrodes, which may be used in one or more processing operations (e.g., cell lysis) on the sensor or portion thereof. The electronics unit may comprise one or more controllers, which may be configured to automate one or more of the processes described herein (e.g., stool sample processing, lysis, isolation or extraction of a biomolecule, storage, analysis, etc.). An example controller system of an electronics unit is described in Example 6 below.

The systems may also comprise one or more beads, as described herein. The beads may be a solid or semi-solid particle. The bead may be a polymeric bead comprising one or more polymers. The gel bead may include a polymer matrix (e.g., matrix formed by polymerization or cross-linking) or an interpenetrating network of polymers. One or more polymers of the bead may be randomly arranged, such as in random copolymers, and/or have ordered structures, such as in block copolymers. The bead may comprise a macromolecule or may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be a solid particle (e.g., a metal-based particle including but not limited to iron oxide, gold or silver) covered with a coating comprising one or more polymers. The bead may be a silica particle. The bead may comprise one or more materials, e.g., metal and silica (e.g., glass), ceramic, etc. The bead may be custom-built or commercially available. In some instances, the bead may have a diameter of between 10 nanometers and 10 micrometers. A range of bead sizes may be utilized in each embodiment. Alternatively, the bead may have a diameter below 10 nanometers or above 10 micrometers. The bead may be configured to capture a biomolecule (e.g., nucleic acids, proteins, lipids, carbohydrates) or a non-biological particle (e.g., metals, small molecules, ions, etc.).

### Computer Systems

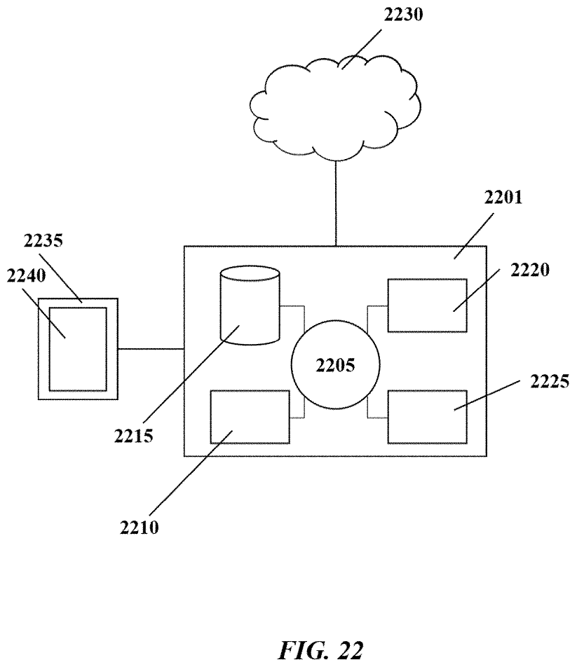

The present disclosure provides computer systems that are programmed to implement methods of the disclosure. FIG. 22 shows a computer system 2201 that is programmed or otherwise configured to analyze a parameter of stool sample collection. The computer system 2201 can regulate various aspects of sample collection of the present disclosure, such as, for example, monitoring completion of stool sample collection, receiving and/or storing an input from one or more sensors, etc. The computer system 2201 can be an electronic device of a user or a computer system that is remotely located with respect to the electronic device. The electronic device can be a mobile electronic device.

The computer system 2201 includes a central processing unit (CPU, also “processor” and “computer processor” herein) 2205, which can be a single core or multi core processor, or a plurality of processors for parallel processing. The computer system 2201 also includes memory or memory location 2210 (e.g., random-access memory, read-only memory, flash memory), electronic storage unit 2215 (e.g., hard disk), communication interface 2220 (e.g., network adapter) for communicating with one or more other systems, and peripheral devices 2225, such as cache, other memory, data storage and/or electronic display adapters. The memory 2210, storage unit 2215, interface 2220 and peripheral devices 2225 are in communication with the CPU 2205 through a communication bus (solid lines), such as a motherboard. The storage unit 2215 can be a data storage unit (or data repository) for storing data. The computer system 2201 can be operatively coupled to a computer network (“network”) 2230 with the aid of the communication interface 2220. The network 2230 can be the Internet, an internet and/or extranet, or an intranet and/or extranet that is in communication with the Internet. The network 2230 in some cases is a telecommunication and/or data network. The network 2230 can include one or more computer servers, which can enable distributed computing, such as cloud computing. The network 2230, in some cases with the aid of the computer system 2201, can implement a peer-to-peer network, which may enable devices coupled to the computer system 2201 to behave as a client or a server.

The CPU 2205 can execute a sequence of machine-readable instructions, which can be embodied in a program or software. The instructions may be stored in a memory location, such as the memory 2210. The instructions can be directed to the CPU 2205, which can subsequently program or otherwise configure the CPU 2205 to implement methods of the present disclosure. Examples of operations performed by the CPU 2205 can include fetch, decode, execute, and writeback.

The CPU 2205 can be part of a circuit, such as an integrated circuit. One or more other components of the system 2201 can be included in the circuit. In some cases, the circuit is an application specific integrated circuit (ASIC).

The storage unit 2215 can store files, such as drivers, libraries and saved programs. The storage unit 2215 can store user data, e.g., user preferences and user programs. The computer system 2201 in some cases can include one or more additional data storage units that are external to the computer system 2201, such as located on a remote server that is in communication with the computer system 2201 through an intranet or the Internet.

The computer system 2201 can communicate with one or more remote computer systems through the network 2230. For instance, the computer system 2201 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 2201 via the network 2230.

Methods as described herein can be implemented by way of machine (e.g., computer processor) executable code stored on an electronic storage location of the computer system 2201, such as, for example, on the memory 2210 or electronic storage unit 2215. The machine executable or machine readable code can be provided in the form of software. During use, the code can be executed by the processor 2205. In some cases, the code can be retrieved from the storage unit 2215 and stored on the memory 2210 for ready access by the processor 2205. In some situations, the electronic storage unit 2215 can be precluded, and machine-executable instructions are stored on memory 2210.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 2201, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 2201 can include or be in communication with an electronic display 2235 that comprises a user interface (UI) 2240 for providing, for example, information on stool sample collection. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 2205. The algorithm can, for example, determine parameters of stool collection (e.g., time course of sample collection), frequency of collection, analysis of the stool sample, etc.

### EXAMPLES

### Example 1—Microfluidic Assay Proof of Concept

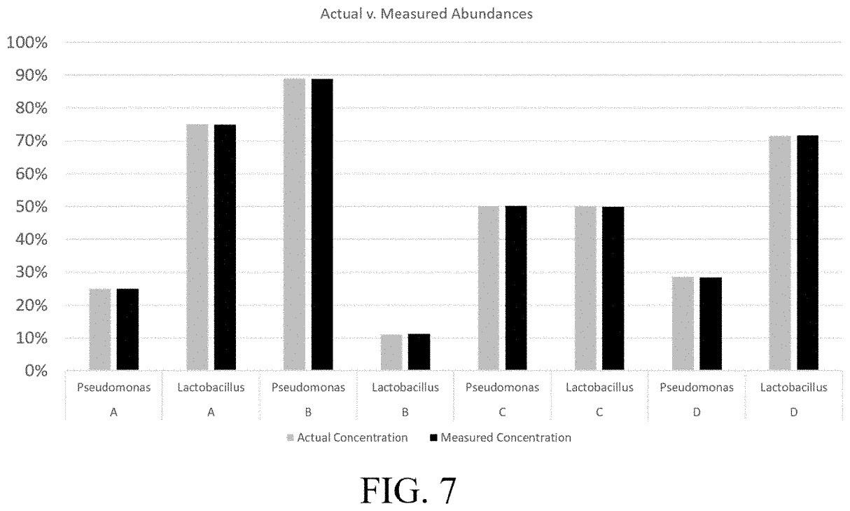

The sensor, which may comprise a microfluidic device, can be tested to determine a parameter of the stool sample, e.g., the abundance of a bacterial strain. Simulated stool samples may be mixed with a defined concentration of a microbe, e.g., Pseudomonas, Lactobacillus. The simulated stool sample may then be processed and analyzed, as described herein. The simulated stool sample may be filtered through the microfluidic device, and the filtered sample may be subjected to lysis. The lysed stool sample may then be subjected to purification, e.g., using magnetic beads. The purified product may then be isolated using the microfluidic device. Presence of bacteria may be detected, e.g., using optical methods or biochemical methods (e.g., sequencing). FIG. 7 shows example data of the measured abundance of control genomic DNA representative of species potentially found in stool. A, B, C, and D represent experimental replicates of samples that are spiked with differing but known concentrations of Pseudomonas or Lactobacillus genomic DNA. Samples are also spiked with a control sequence of DNA of known and consistent quantity, which may be used to quantitate and normalize raw data. The y-axis corresponds to the range of quantitation for this assay, e.g., 100% corresponds to the approximate concentration of DNA that would result in a saturated signal, above which all signal would appear to be the same intensity. The spiked samples are processed via a hybridization assay intended to screen for the quantity of microbe specific DNA. The concentration of DNA is then imaged via fluorescent microscopy and quantified using pixel intensity measurements, i.e. densitometry. The ‘actual concentration’ bars refer to the normalization of known DNA to intensity of the spiked control sequence. The ‘measured concentration’ group refers to the normalization of intensity of a sample to the intensity of the spiked control sequence and anchored to the first sample in a series, i.e., sample A. This results in ‘actual’ and ‘measured’ quantities being identical for each A sample and allows examination of the variation in the assay in samples B, C & D.

### Example 2—Abundance of Pseudomonas and Lactobacillus in a Simulated Stool Sample

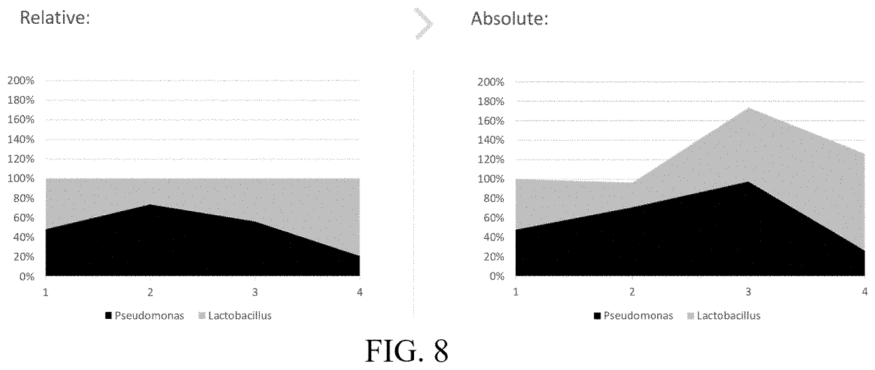

The sensor comprising a microfluidic device can provide information on the abundance of a microbe in a sample. Simulated stool samples may be mixed with a defined concentration of a microbe, e.g., pseudomonas, lactobacillus. The simulated stool sample may then be processed and analyzed, as described herein. The simulated stool sample may be filtered through the microfluidic device, and the filtered sample may be subjected to lysis. The lysed stool sample may then be subjected to purification, e.g., using magnetic beads. The purified product may then be isolated using the microfluidic device. Presence of bacteria may be detected, e.g., using optical methods or biochemical methods (e.g., sequencing). FIG. 8 shows example data of the measured abundance of control genomic DNA representative of species potentially found in stool. Samples 1, 2, 3, & 4 represent experimental replicates of samples that are spiked with differing but known concentrations of Pseudomonas or Lactobacillus genomic DNA. Samples are also spiked with a control sequence of DNA of known and consistent quantity, this is used to quantitate and normalize raw data. The y-axis corresponds to the total abundance of DNA in a sample, i.e. combination of Pseudomonas and Lactobacillus DNA signal. The spiked samples are processed via a hybridization assay intended to screen for the quantity of microbe specific DNA. The concentration of DNA is then imaged via fluorescent microscopy and quantified using pixel intensity measurements, i.e. densitometry. Without normalization to spiked controls only the relative abundance can be measure. Relative abundance can only provide information on how the amount of microbial DNA differs compared to other DNA signals, (e.g. FIG. 8 left panel). However, with spiked-in controls and intensity normalization this assay is capable of detecting changes in absolute abundance, (e.g. FIG. 8 right panel).

### Example 3—Sterilization Protocol for Preventing Cross Contamination Between Samples

The collection unit, as described herein, may be used to automatically perform a plurality of processes, e.g., lysis of cells within a stool sample and isolation or extraction of a biomolecule. Sterilization may be performed in the collection unit (e.g., the lysis unit or the isolation or extraction unit) to prevent cross-contamination between samples. Sterilization may comprise the use of a sterilization agent (e.g., bleach solution), which may be introduced in the fluid lines and/or chambers of the collection unit.

To test the effectiveness of a sterilization protocol, two or more stool samples can be prepared, each with a known concentration of microbes that are spiked in. Simulated stool samples may be mixed with a defined concentration of a microbe, e.g., Pseudomonas, Lactobacillus, Escherichia, Shigella, Enterococcus, Romboutsia, Faecalibacterium, Blautia, Streptococcus, Bacteroides, Lachnospiracea, etc. The simulated stool sample may then be processed and analyzed, as described herein (e.g., using a system architecture, or a portion thereof shown in FIGS. 15A-C). Nucleic acid molecules (e.g., DNA) may be isolated or extracted from the stool samples using silica magnetic beads. The beads, or the nucleic acid molecules attached thereto, may be subjected to sequencing (e.g., 16(S) sequencing). The nucleic acid molecules on the beads may then be analyzed to determine the microbes in each sample. After each sample processing and isolation or extraction of the DNA, the system (e.g., the collection unit), or portion thereof, may be subjected to sterilization (e.g., using a bleach solution).

FIG. 19 shows example data of the measured abundance of genomic DNA in two simulated stool samples. Stool #1A and Stool #1B are aliquots of the same sample comprising a mixture of microbes. The biculture sample is a simulated stool sample containing only two strains of microbes. To test the effectiveness of the sterilization protocol, Stool #1A is introduced into the system and processed (e.g. subjected to lysis and isolation or extraction of DNA using silica magnetic beads). The isolated DNA is subjected to sequencing (e.g., using 16(S) sequencing), and the system (e.g., the collection unit) is sterilized. The biculture sample is then introduced into the system and processed. The isolated DNA from the biculture sample is subjected to sequencing, and the system is sterilized again. Next, Stool #1B is introduced into the system and process. The isolated DNA from the Stool #1B sample is sequenced. Statistical analysis (e.g., intraclass correlation, ICC) may be used to measure variability of the sequencing results.

As shown in FIG. 19, the Stool #1A and the Stool #1B samples contain similar levels of DNA percentages from each of the spiked in microbe types, despite the biculture sample having been introduced in between the introduction of the Stool #1A and Stool #1B samples. Similarly, the biculture sample exhibits high DNA percentages of the two microbes that are used to generate the biculture sample, indicating little cross-contamination from Stool #1A. Stool #1B, introduced after introduction of the biculture sample (and subsequent sterilization), also exhibits low percentages of the strains present in the biculture. Overall, these results support that sterilization between sample processing runs are effective in minimizing cross-contamination.

### Example 4—DNA Isolation and Sequencing from Known Microbial Samples

Similar to Example 1, the accuracy of DNA isolation in a system described herein (e.g., collection unit with multiple processing operations) may be measured. Simulated stool samples (or simulated samples comprising known microbial communities) may be mixed with a defined concentration of a microbe, e.g., Pseudomonas, Lactobacillus, Escherichia, Shigella, Enterococcus, Romboutsia, Faecalibacterium, Blautia, Streptococcus, Bacteroides, Lachnospiracea. The simulated stool sample may then be processed and analyzed, as described herein (e.g., using a system architecture, or a portion thereof shown in FIGS. 15A-C). Nucleic acid molecules (e.g., DNA) may be isolated or extracted from the stool samples using silica magnetic beads. The beads, or the nucleic acid molecules attached thereto, may be subjected to sequencing (e.g., 16(S) sequencing). The nucleic acid molecules on the beads may then be analyzed to determine the microbes in each sample.

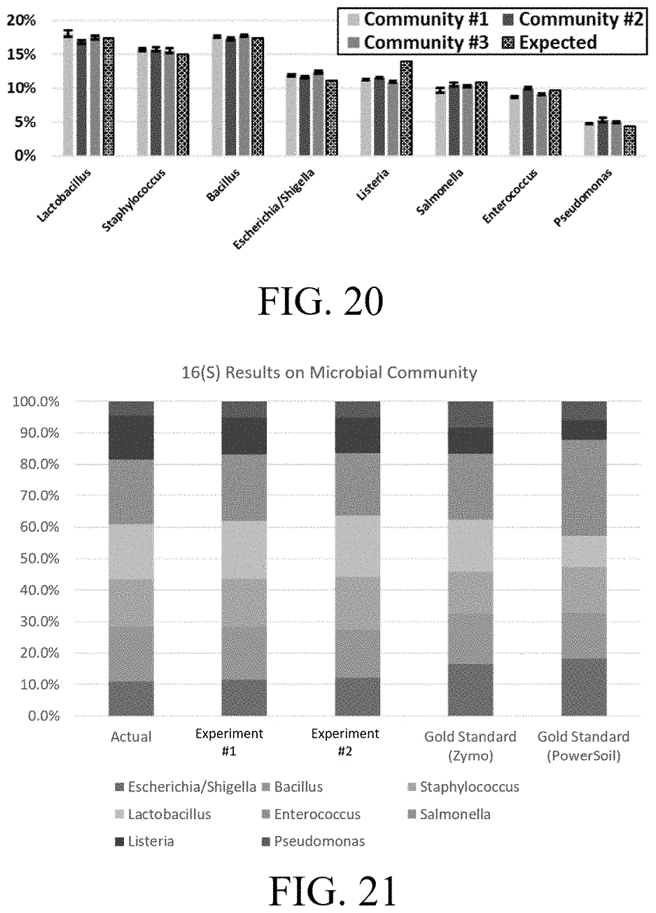

FIG. 20 shows example data of the measured abundance of genomic DNA isolated from the beads and subjected to sequencing. Communities 1, 2, and 3 represent experimental replicates of a sample that is spiked with known concentrations of microbes (or microbial DNA). The percentage of DNA from each microbe type for each experimental replicate is compared to the expected abundance (based on the spike-in concentrations). The y-axis corresponds to the percentage of DNA measured by sequencing, and the x-axis corresponds to the different microbe types. As shown in FIG. 20, the percentage of DNA from each of the tested microbe types (Lactobacillus, Staphylococcus, Bacillus, Escherichia/Shigella, Listeria, Salmonella, Enterococcus, and Pseudomonas) is similar to the expected percentage.

FIG. 21 shows example data of the measured abundance of genomic DNA isolated from the beads and subjected to sequencing, compared to two gold-standard techniques. Experiment #1 and Experiment #2 represent experimental replicates of a sample that is spiked with known concentrations of microbes (or microbial DNA). The percentage of DNA from each microbe type for each experimental replicate is compared to the actual abundance (based on the spike-in concentrations). The y-axis corresponds to the percentage of DNA measured by sequencing, and the x-axis corresponds to different measurement techniques. As shown in FIG. 20, the percentage of DNA from each of the tested microbe types (Lactobacillus, Staphylococcus, Bacillus, Escherichia/Shigella, Listeria, Salmonella, Enterococcus, and Pseudomonas) is similar to the actual percentage and matches the results from other gold standard techniques. Overall, these results indicate that isolation of genomic DNA using the lysis, isolation, and analysis procedures described herein produces accurate results of the microbe communities in a simulated stool sample.

### Example 5—DNA Isolation Protocol

As described herein, a biomolecule, such as one or more DNA molecules, may be isolated from a stool sample or derivative thereof (e.g., lysed stool sample). An example DNA isolation protocol is provided below, which may be performed by a user or may be performed automatically using, for instance, the collection unit.

Materials. Example materials include, but are not limited to: filtered Binding buffer: 1-part 4M guanidine thiocyanate, 40 mM Tris, 17.6 mM EDTA, pH 8.0, 1-part 100% IPA; Filtered Wash buffer: 10 mM TAE (10 mM Tris Acetate, 1 mM EDTA) buffer, pH 4.5; Filtered Elution buffer: 10 mM TE (10 mM Tris, 1 mM EDTA) buffer, pH 8; SeraSil-Mag 400 silica magnetic beads (Cytiva, Cat #29357371); Round bottom 96-well plate with lid (Greiner Bio-One, Cat #650180); 96 Deep Well Plate (Thermo Scientific, Cat #: 2602051); Magnetic plate for round-bottom 96-well plates (EpiGenteck, EpiMag); 0.22 μm PES Membrane Filter Unit (Millipore, Cat #SLGPR33RS).

Example Procedure: Magnetic Bead Preparation. 1. Before you begin, ensure you have enough filtered buffer stock for all isolation steps and record the lot numbers of the following solutions on your experiment sheet. i. SeraSil-Mag 400 silica magnetic bead stock ii. Binding buffer, wash buffer, elution (TE) buffer. 2. Add total volume of magnetic bead stock that will be used into an appropriately sized vial and place the vial next to a magnet. Remove supernatant. (50 μL/sample, e.g. 10 samples will require total volume of 500 μL of magnetic beads). 3. Clean the bead stock by adding 150 μL of elution buffer per 50 of bead stock and resuspend. 4. Capture the beads with a magnet and remove supernatant. Repeat this process 2x. Add 75 μL of binding buffer and 75 μL of 100% IPA to the magnetic beads for every 50 μL of bead stock (e.g. 750 μL of binding buffer for 10 samples). 5. Add 150 μL of the magnetic bead preparation and 50 μL of sample into 96 deep well plate (Thermo Scientific, Cat #: 2602051). Pipette mixture up and down at least 5×, or until the bead/sample mixture appears homogenous. 6. Place on the plate on a plate shaker at 600 rpm for 5 minutes. 7. Place the sample plate on the magnetic plate (EpiMag HT, Cat #Q10002-1) to immobilize the magnetic beads, wait at least 30 seconds for beads to accumulate. 8. Remove the supernatant with a multichannel pipette. 9. While keeping samples on the magnetic plate, gently multichannel pipette 200 of wash buffer against the well wall opposite of the beads and immediately pipette out buffer and discard. Remove any remaining drops that have settled to the bottom of the vial. Repeat 2×. i. Note: Use very slow dispensing and aspiration to prevent bead resuspension. The magnet is not powerful enough to prevent all resuspension. ii. After removing fluid from second wash be sure to look at all wells for residual fluid. A third round of wash buffer removal may be necessary with fresh pipettes for these residuals. 10. Add 50 μL of elution buffer to beads with a multichannel pipette. i. Note: Ensure the 96-well plate is off of the magnet plate. It will be necessary to pipette directly on the beads to resuspend. 11. Place on plate shaker at 600 speed for 10 minutes. 12. Place plate on magnet and using a multichannel pipette, transfer supernatant to a round bottom 96 well plate with lid. Apply adhesive microseal B film.

### Example 6—Electronics Comprising Micro-Controller Units for Automation of Stool Sample Processing

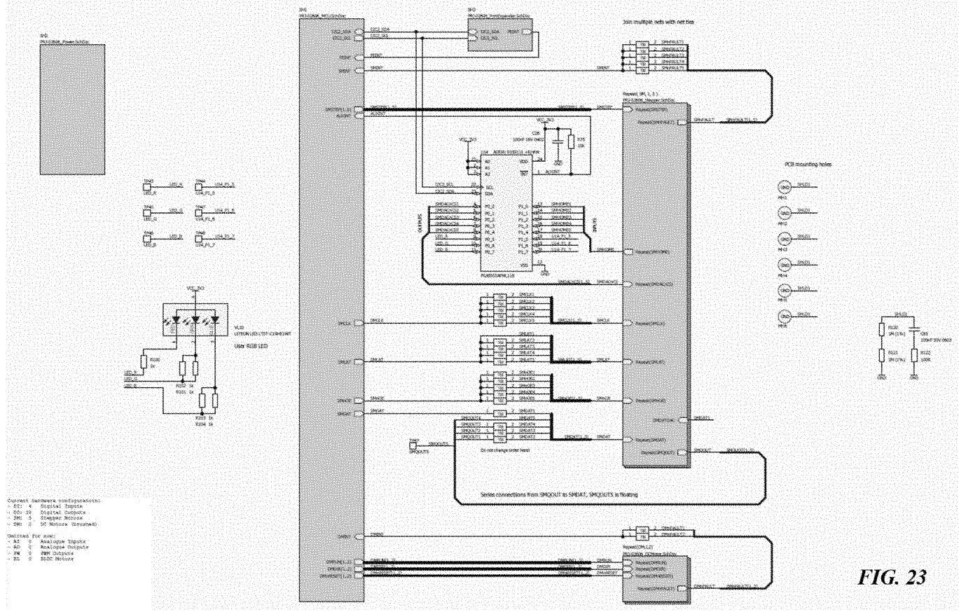

As described herein, one or more processes may be performed automatically. In such cases, the automated processes may be controlled using one or more micro-controllers (e.g., comprised in an electronics unit). For instance, the system (e.g., collection unit, or component thereof) may comprise a main controller which comprises integrated circuitry that can control, for example, input or output distribution and all major communication between the micro-controller and any electrical components or peripheral items. An example main controller architecture is schematically illustrated in FIG. 23.

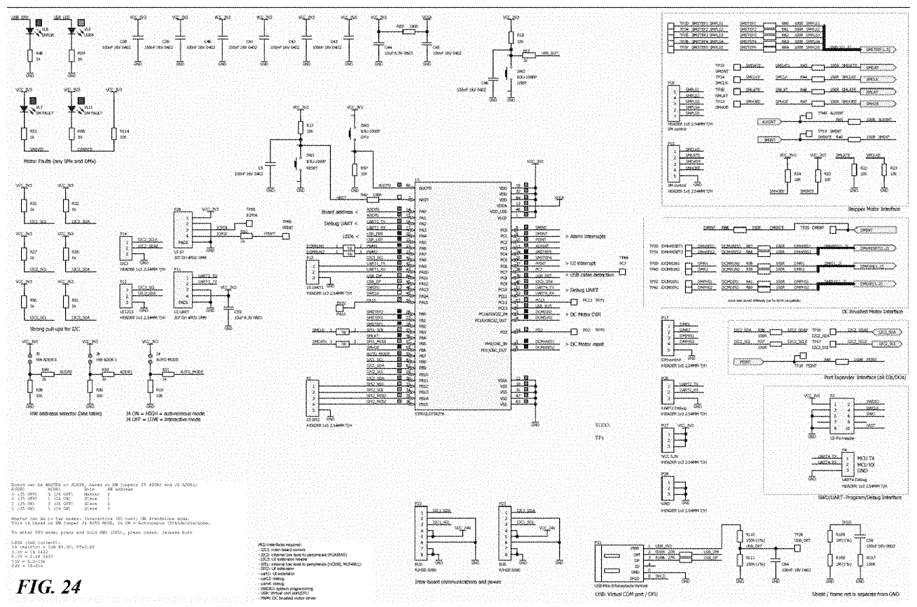

The system may additionally comprise a controller, such as a microcontroller unit (MCU). The MCU may be, for instance, a low-power 32-bit microcontroller and may comprise a motor controller interface, inter-board communications, input and output distribution, hardware headers and a communication interface (e.g., a USB COM port interface). An example of such an MCU unit is schematically shown in FIG. 24.

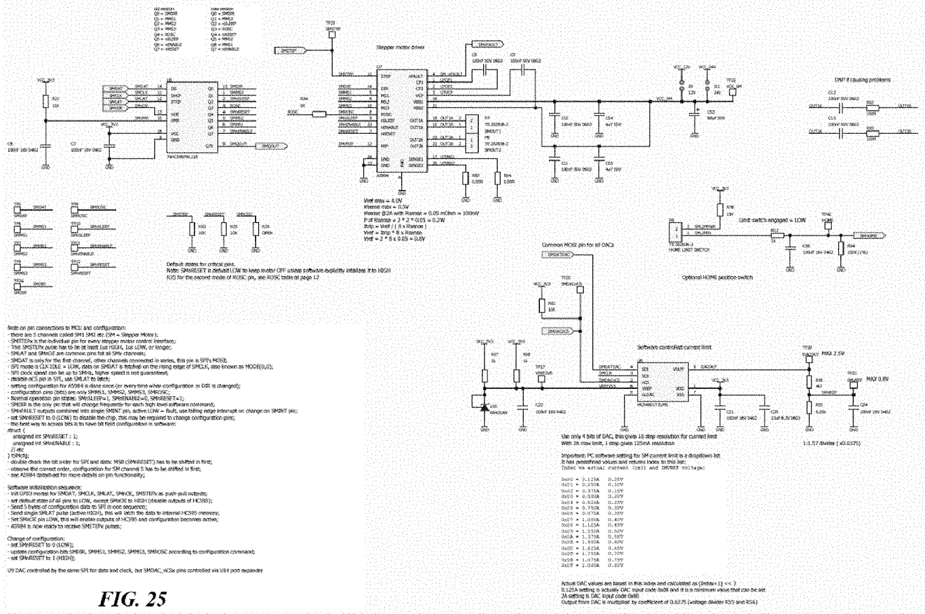

The system may comprise a controller comprising a stepper driver, optionally with power interfaces and an MCU interface for detail and firmware notes. An example of such a controller is schematically shown in FIG. 25.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1-76. (canceled)

77. A method for processing a stool sample, comprising:
(a) receiving said stool sample at a first location, wherein said stool sample is collected at a second location different than said first location, and wherein said second location is within 1 mile of said first location; and
(b) at said first location, automatically processing said stool sample to extract or isolate at least one biomolecule from said stool sample.

78. The method of claim 77, further comprising transmitting said at least one biomolecule of (b) or derivative thereof to a third location different than said first location and said second location, wherein at said third location, said at least one biomolecule or derivative thereof is analyzed to identify at least a portion of said at least one biomolecule.

79. The method of claim 77, further comprising, analyzing said at least one biomolecule at said first location.

80. The method of claim 79, wherein said at least one biomolecule comprises a deoxyribonucleic acid (DNA) molecule, and wherein said analyzing comprises sequencing said DNA molecule.

81. The method of claim 77, wherein (b) comprises using one or more beads to extract or isolate said at least one biomolecule.

82. The method of claim 77, wherein said stool sample is received and processed in a collection unit.

83. The method of claim 82, wherein said collection unit comprises a sensing unit comprising a sensor and an electronics unit.

84. The method of claim 83, further comprising using said sensor to analyze said at least one biomolecule.

85. The method of claim 83, wherein said electronics unit of said sensing unit comprises a communication interface for transmitting data of said stool sample or derivative thereof to an electronic device in communication with said communication interface.

86. The method of claim 82, further comprising automatically cleaning said collection unit.

87. The method of claim 82, wherein said collection unit comprises a storage unit.

88. The method of claim 87, wherein said storage unit comprises a multi-well unit, and wherein said at least one biomolecule is extracted or isolated in a well of said multi-well unit.

89. The method of claim 87, further comprising, storing said at least one biomolecule in said storage unit.

90. The method of claim 82, wherein said collection unit comprises an array, and wherein said at least one biomolecule is extracted or isolated using said array.

91. The method of claim 90, further comprising analyzing said at least one biomolecule on said array.

92. The method of claim 77, wherein (b) comprises homogenizing said stool sample.

93. The method of claim 77, wherein (b) comprises lysis of one or more cells in said stool sample.

94. The method of claim 93, wherein said lysis is performed using one or more members selected from the group consisting of ultrasonic lysis, mechanical lysis, biological lysis, and chemical lysis.

95. The method of claim 94, wherein said lysis comprises mechanical lysis, wherein said mechanical lysis is performed using beads.

96. The method of claim 77, wherein (b) comprises filtering said stool sample or derivative thereof.
